SEQUENCE LISTING (7 sequences)

SEQ ID NO: 1 - DNA, 42 bp, Artificial Sequence
  Description of Artificial Sequence Synthetic oligonucleotide
tttttggccgcacgcgtccaccatggggtacaactacttttt

SEQ ID NO: 2 - DNA, 42 bp, Artificial Sequence
  Description of Artificial Sequence Synthetic oligonucleotide
tttttgtagttgtaccccatggtggacgcgtgcggccttttt

SEQ ID NO: 3 - protein, 15 aa, Artificial Sequence
  Description of Artificial Sequence Synthetic peptide
EPPLSQEAFADLWKK

SEQ ID NO: 4 - protein, 13 aa, Artificial Sequence
  Description of Artificial Sequence Synthetic peptide
PESQEAFADLWKK

SEQ ID NO: 5 - protein, 11 aa, Artificial Sequence
  Description of Artificial Sequence Synthetic peptide
SQEAFADLWKK

SEQ ID NO: 6 - protein, 25 aa, Artificial Sequence
  Description of Artificial Sequence Synthetic peptide
PESQEAFADLWPESQEAFADLWKKK

SEQ ID NO: 7 - protein, 9 aa, Artificial Sequence
  Description of Artificial Sequence Synthetic peptide
SQEAFADLW

(19) United States
(12) Patent Application Publication
(10) Pub. No.: US 2004/0266843 A1
(43) Pub. Date: Dec. 30, 2004

(54) Sulfonamide substituted indolinones as inhibitors of DNA dependent protein kinase (DNA-PK)

(75) Inventors: Anthony R. Howlett, Berkeley, CA (US); Audie Rice, Burlingame, CA (US); Deborah Moshinsky, Cambridge, MA (US); Ola Hammarsten, Kungsbacka (SE)

Correspondence Address: FOLEY AND LARDNER, SUITE 500, 3000 K STREET NW, WASHINGTON, DC, 20007, US

(73) Assignee: Sugen, Inc.

(21) Appl. No.: 10/793,943

(22) Filed: Mar. 8, 2004

Related U.S. Application Data
(60) Provisional application No. 60/452,549, filed on Mar. 7, 2003.

(51) Int. Cl. A61K 31/427; A61K 31/404

(52) U.S. Cl. 514/365; 514/414

(57) ABSTRACT

The present invention relates generally to the field of radiosensitizing agents which are capable of enhancing radiotherapy by inhibiting DNA-PK (DNA-protein kinase). In particular, it relates to sulfonamide substituted indolinones which inhibit DNA-PK.

### CROSS-REFERENCE TO RELATED APPLICATION

The present application claims priority to U.S. Provisional Application No. 60/452,549, filed Mar. 7, 2003, the contents of which are hereby incorporated by reference in their entirety and for all purposes.

### FIELD OF THE INVENTION

The present invention relates generally to the field of radiosensitizing agents which are capable of enhancing radiotherapy by inhibiting DNA-PK (DNA-protein kinase). In particular, sulfonamido-substituted indolinone compounds of Formula (I), (II), (III), (IV), (V), and (VI) are useful as inhibitors of DNA-PK as well as inhibitors of certain protein tyrosine kinases, serine/threonine kinases and cellular tyrosine kinases.

### BACKGROUND OF THE INVENTION

Radiation therapy is used in more than one third of all cancer patients. Radiation therapy, also called radiotherapy, is the treatment of cancer and other diseases with ionizing radiation. Ionizing radiation deposits energy that injures or destroys cells in the area being treated (the “target tissue”) by damaging their genetic material, making it impossible for these cells to continue to grow. Radiotherapy is successful because ionizing radiation kills dividing cells and is thus slightly more toxic to fast growing cancer cells. Radiotherapy may be used to treat localized solid tumors, such as cancers of the skin, tongue, larynx, brain, breast, or uterine cervix. It can also be used to treat leukemia and lymphoma (cancers of the blood-forming cells and lymphatic system, respectively).

One type of radiation therapy commonly used involves photons, “packets” of energy. X-rays were the first form of photon radiation to be used to treat cancer. Depending on the amount of energy they possess, the rays can be used to destroy cancer cells on the surface of or deeper in the body. The higher the energy of the x-ray beam, the deeper the x-rays can go into the target tissue. Linear accelerators and betatrons are machines that produce x-rays of increasingly greater energy. The use of machines to focus radiation (such as x-rays) on a cancer site is called external beam radiotherapy.

Gamma rays are another form of photons used in radiotherapy. Gamma rays are produced spontaneously when certain elements (such as radium, uranium, and cobalt 60) release radiation as they decompose, or decay. Each element decays at a specific rate and gives off energy in the form of gamma rays and other particles. X-rays and gamma rays have the same effect on cancer cells.

Another technique for delivering radiation to cancer cells is to place radioactive implants directly in a tumor or body cavity. This is called internal radiotherapy. (Brachytherapy, interstitial irradiation, and intracavitary irradiation are examples of internal radiotherapy.) In this treatment, the radiation dose is concentrated in a small area, and the patient stays in the hospital for a few days. Internal radiotherapy is frequently used for cancers of the tongue, uterus, and cervix.

Several new approaches to radiation therapy are being evaluated to determine their effectiveness in treating cancer. One such technique is intraoperative irradiation, in which a large dose of external radiation is directed at the tumor and surrounding tissue during surgery.

Another investigational approach is particle beam radiation therapy. This type of therapy differs from photon radiotherapy in that it involves the use of fast-moving subatomic particles to treat localized cancers. A sophisticated machine is needed to produce and accelerate the particles required for this procedure. Some particles (neutrons, pions, and heavy ions) deposit more energy along the path they take through tissue than do x-rays or gamma rays, thus causing more damage to the cells they hit. This type of radiation is often referred to as high linear energy transfer (high LET) radiation.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

Other recent radiotherapy research has focused on the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells. The success of this technique will depend upon both the identification of appropriate radioactive substances and determination of the safe and effective dose of radiation that can be delivered in this way.

Radiation therapy may be used alone or in combination with chemotherapy or surgery. Like all forms of cancer treatment, radiation therapy can have side effects. Possible side effects of treatment with radiation include temporary or permanent loss of hair in the area being treated, skin irritation, temporary change in skin color in the treated area, and tiredness. Other side effects are largely dependent on the area of the body that is treated.

As stated above, administration of a radiosensitizers prior to radiation therapy makes the tumor cells more likely to be damaged. To that end, a variety of radiosensitizing agents have been developed. The primary focus of the present invention is to provide an agent which sensitizes cancer cells to radiation therapy.

Several lines of evidence now indicate that the tumor killing effect of radiation is mediated by induction of DNA double stranded breaks (DSBs). In human cells, DSBs introduced by ionizing radiation are predominantly repaired through a non-homologous end-joining pathway that requires the function of the ligase Iv/xrcc4 complex and the DNA dependent protein kinase (DNA-PK). DNA-PK is a serine-threonine protein kinase composed of two components. The sequence of DNA-PK has been determined and has been indexed as accession no. P78527 (Swissprot) which can be found at the following: http://www.ncbi.nlm.nih.gov/entrez/query.fcgi?cmd=Retrieve&db=protein&list_uids=13633184&dopt=GenPept. One is a catalytic subunit of 460 kDa (DNA-PKcs), which shares a domain of homology with members of the phosphatidylinositol 3-kinase (PI3-kinase) family of lipid kinases. The other component, Ku, functions as a DNA-binding subunit and is a heterodimer of 70 and 86 kDa subunits. Ku binds specifically to DSB via a preformed channel and recruits DNA-PKcs. When the full DNA-PK complex is assembled on DNA-ends the kinase is activated. Several lines of evidence indicate that activation occurs when the single-stranded 3′- and 5′-ends of the DSB is threaded into an enclosed channel present in DNA-PKcs. Recently it was shown that DNA-PK phosphorylation specifically regulates the activity of artemis, a nuclease required for repair of DSBs. It is therefore possible that the activation of DNA-PK by DSBs is required for processing of the DNA-ends in preparation for DSB-repair.

Animals with targeted deletions of DSB-repair genes are now available. The common phenotypes among these knock-out mice are a 5-10 fold increased sensitivity to ionizing radiation, defect in DSB-repair and absence of B and T-cells because of a developmental block in V(D)J-recombination. In addition to these defects, Ku70, Ku80, xrcc4 and ligase IV mutant animals also show neurological defects, premature cell senecense, growth retardation and a general susceptibility to cancer development. In contrast, DNA-PKcs knock-out animals have no additional defects and develop cancer only from the lymphoid tissues secondary to the V(D)J-recombination defect. In fact, in a skin cancer induction assay, DNA-PKcs deficient mice were resistant to cancer development due to rapid death of cells with DNA damage. Based on this knowledge, radiosensitization by transient inhibition of DNA-PK is not expected to give specific side effects or increased cancer susceptibility. In addition, there exists evidence that DNA-PK is overexpressed in tumor cells resulting in resistance to treatment. A specific DNA-PK inhibitor is therefore not expected to be toxic and could selectively sensitize tumor cells to ionizing radiation. DNA-PK is therefore a reasonable target for development of radiosensitizers.

Loss of the function of the DNA-dependent protein kinase (DNA-PK) results in increased sensitivity to ionizing radiation due to inability to repair DNA double-strand breaks. In addition, over-expression of DNA-PK in tumor cells results in resistance to ionizing radiation and anti-cancer therapy. It is therefore possible that inhibition of DNA-PK will enhance the preferential killing of tumor cells by radiotherapy. Available inhibitors of DNA-PK, like wortmannin, display 100-fold higher activity against p 110, a phoshatidylinositol-3-kinase, resulting in cell toxicity.

Certain PI3 inhibitors also inhibit DNA-PK and ATM. One member of this group, wortmannin, has been shown to inhibit DSB-repair and result in a 2-5 fold sensitization of cells to ionizing radiation probably through its inhibition of DNA-PK and ATM. A problem with the PI3-kinase inhibitors is that they induce cell cycle arrest and general toxicity because P13-kinase activity is required for many cellular processes including growth factor signaling. This makes it difficult to evaluate the contribution of DNA-PK-inhibition to the radiosensitization process. Thus, at concentrations required to observe radiosensitization, wortmannin induces general toxicity that limits its use as a radiosensitizer in patients.

Wortmannin has been shown to be an efficient radiosensitizer. Since wortmannin is able to inhibit DNA-dependent protein kinase and double stranded break rejoining, it is believed that its mechanism of radiation sensitization is through the inhibition of DNA-PK mediated repair of DSBs. However, wortmannin has been shown to be a non-competitive inhibitor, see Cancer Research, vol. 59, pages 2581-2586 (1999). Thus, a need exists to develop more specific inhibitors of DNA-PKcs for use as radiosensitizing agents.

### SUMMARY OF THE INVENTION

The present inventors have found that 5-sulfonamido substituted indolinones are specific DNA-PK inhibitors. Inhibition kinetics and a direct assay for ATP-binding showed that these compounds inhibit DNA-PK by ATP competition. The 5-sulfonamido substituted indolinones inhibited DNA-PK in cells since it inhibited DNA double-strand break repair in cells resulting in an increase sensitization to ionizing radiation.

The present invention relates to a method of inhibiting DNA-PK comprising administering to a patient in need of such inhibition, an effective amount of a 5-sulfonamido-substituted indolinone.

In one embodiment of the inventive method, the patient is administered a compound of Formula (I):

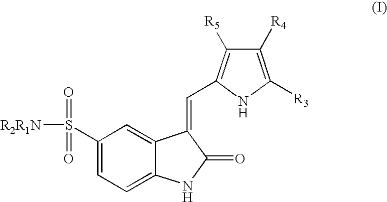

wherein:

R₁ and R₂ are independently selected from the group consisting of H, optionally substituted phenyl, thiazolyl and lower alkyl,

or R₁ and R₂ combine to form an optionally fused heterocyclic ring, which is optionally substituted by —O-alkyl, Br, Cl or F, provided that only one of R₁ and R₂ is alkyl or hydrogen at the same time and further provided that R₁ is not alkyl when R₂ is hydrogen and that R₁ is not hydrogen when R₂ is alkyl;

R₃, R₄ and R₅ are independently selected from the group consisting of H, lower alkyl optionally substituted with hydroxy and —(Y)₀₋₁—Y₁,

or R₃ and R₄ may combine to form a cyclic 6-membered alicyclic ring which may be substituted with one or more lower alkyl, provided that no more than two of R₃, R₄ or R₅ are H at the same time and further provided that at least one of R₃, R₄ or R₅ is —(Y)₀₋₁—Y₁;

Y is —CH₂—, —CH₂—CH₂—, —CH₂—CH₂—CH₂— or —C(O)NHR₆—;

Y₁ is —C(O)OR′, —C(O)NR₆R₇ or —NR₆R₇, where R′ is H or lower alkyl;

R₆ and R₇ are independently selected from the group consisting of H and lower alkyl optionally substituted by —NR₈R₉;

or R₆ and R₇ may combine to form a heterocyclic ring which may include an additional heteroatom selected from the group consisting of N, O and S and which may be further substituted by lower alkyl or hydroxy;

R₈ and R₉ are independently H and lower alkyl;

or R₈ and R₉ may combine to form a heterocyclic ring which may include an additional heteroatom selected from the group consisting of N, O and S and pharmaceutically acceptable salts thereof.

In another embodiment of the inventive method the patient is administered a compound of Formula (II):

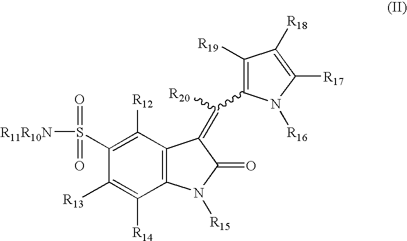

wherein:

R₁₀ and R₁₁ are independently selected from the group consisting of hydrogen, alkyl optionally substituted with amino, hydroxy, a 5-membered to 6-membered heteroalicyclic ring or halo, aryl, heteroaryl, cycloalkyl, alkenyl, alkynyl, heteroalicyclic, or R₁₀ and R₁₁ may combine to form a 5-membered or 6-membered heterocyclic ring which may be optionally fused;

R₁₂, R₁₃ and R₁₄ are independently selected from the group consisting of hydrogen, alkyl, trihaloalkyl, cycloalkyl, alkenyl, alkynyl, aryl, heteroaryl, heteroalicyclic, hydroxy, alkoxy, aryloxy, mercapto, alkylthio, arylthio, sulfinyl, sulfonyl, S-sulfonamido, N-sulfonamido, trihalomethane-sulfonamido, carbonyl, C-carboxy, O-carboxy, C-amido, N-amido, cyano, nitro, halo, O-carbamyl, N-carbamyl, O-thiocarbamyl, N-thiocarbamyl, amino and —NR₂₁R₂₂;

R₁₅ is selected from the group consisting of hydrogen, alkyl, alkenyl, alkynyl, cycloalkyl, aryl, hydroxy, alkoxy, C-carboxy, O-carboxy, acetyl, C-amido, C-thioamido, sulfonyl and trihalomethanesulfonyl;

R₂₀ is selected from the group consisting of hydrogen, halo, alkyl, cycloalkyl, aryl, heteroaryl and heteroalicyclic;

R₂₁ and R₂₂ are independently selected from the group consisting of hydrogen, alkyl, cycloalkyl, aryl, carbonyl, acetyl, sulfonyl, trifluoromethanesulfonyl and, combined, a five- or six-member heteroalicyclic ring;

R₁₃ and R₁₄ may combine to form a six-member aryl ring, a methylenedioxy group or an ethylenedioxy group;

R₁₆ is selected from the group consisting of hydrogen, alkyl, cycloalkyl, alkenyl, alkynyl, aryl, heteroaryl, heteroalicyclic, hydroxy, alkoxy, aryloxy, carbonyl, acetyl, C-amido, C-thioamido, amidino, C-carboxy, O-carboxy, sulfonyl and trihalomethane-sulfonyl;

R₁₇, R₁₈ and R₁₉ are independently selected from the group consisting of hydrogen, alkyl, trihaloalkyl, cycloalkyl, alkenyl, alkynyl, aryl, heteroaryl, heteroalicyclic, hydroxy, alkoxy, aryloxy, mercapto, alkylthio, arylthio, sulfinyl, sulfonyl, S-sulfonamido, N-sulfonamido, carbonyl, C-carboxy, O-carboxy, cyano, nitro, halo, O-carbamyl, N-carbamyl, O-thiocarbamyl, N-thiocarbamyl, C-amido, N-amido, amino, —NR₂₁R₂₂ and -(alk₁)Z wherein Alk₁ is selected from the group consisting of alkyl, alkenyl or alkynyl; and, Z is a polar group;

or R₁₇ and R₁₈ or R₁₈ and R₁₉ may combine to form an alicyclic ring;

or a pharmaceutically acceptable salt thereof.

In another embodiment of the inventive method the patient is administered a compound of Formula (III):

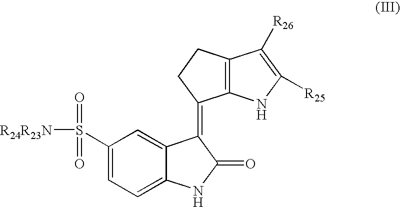

wherein:

R₂₃ and R₂₄ are independently selected from the group consisting of H, optionally substituted phenyl, lower alkyl and cycloalkyl,

or R₂₃ and R₂₄ combine to form an optionally fused heterocyclic ring, which is optionally substituted by —O-alkyl, Br, Cl or F;

R₂₅ and R₂₆ are independently selected from the group consisting of hydrogen, lower alkyl, sulfonyl, —C(O)R₂₇, —C(O)OR₂₇, —C(O)NR₂₇R₂₈, halo, trihaloalkyl, aryl, heteroaryl, wherein R₂₇ and R₂₈ are independently selected from the group consisting of hydrogen, lower alkyl, lower alkyl substituted with one or more of amino, hydroxy, 5-membered to 6-membered heterocyclic ring or R₂₇ and R₂₈ may combine to form a 5-membered to 6-membered heterocyclic ring which may be optionally substituted with alkyl;

or a pharmaceutically acceptable salt thereof.

In another embodiment of the inventive method the patient is administered a compound of Formula (IV), (V) and (VI):

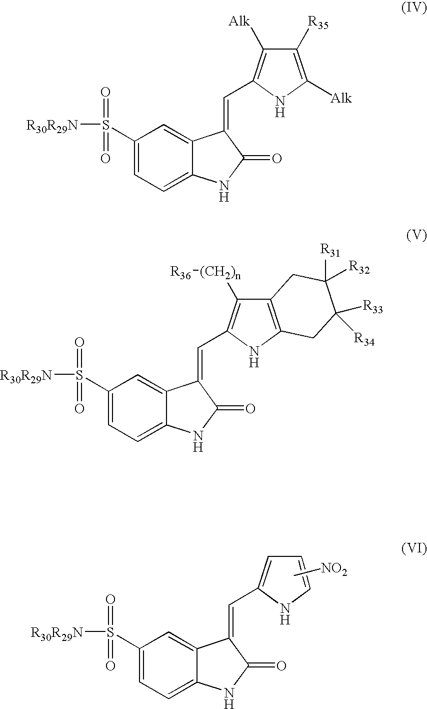

wherein R₂₉-R₃₄ are independently selected from the group consisting of H, lower alkyl and cycloalkyl;

R₃₅ is selected from the group consisting of —CH₂—C(O)—X′—(CH₂)ₙ—R₃₆, —CH₂—R₃₆

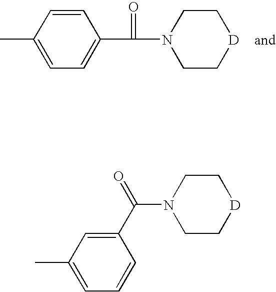

where D is O or N—CH₃;

X′ is NH, S, O or a bond;

R₃₆ is a polar group selected from the group consisting of —C(O)OR′, —C(O)NR₃₇R₃₈, piperazinyl and morpholinyl, Z may be further substituted by —(CH₂)₀₋₁-Z₁, where Z₁ is a polar group selected from the group consisting of —C(O)OR₃₇, —C(O)NR₃₇R₃₈, amino, dialkylamino, hydroxy, piperazinyl, pyrrolidinyl and morpholinyl; when R₃₆ is further substituted, R₃₇ is not present;

R₃₇ and R₃₈ are independently H and lower alkyl;

n is 0-2; and

Alk is lower alkyl of 1-4 carbons;

and pharmaceutically acceptable salts thereof.

In a preferred embodiment, the compound administered is according to formula II, wherein R¹⁰ is phenyl, R₁₂, R₁₃, R₁₄, R₁₅, R₁₆ and R₂₀ are hydrogen, R₁₇ and R₁₉ are ethyl and R₁₈ is a propionic acid moiety.

In a preferred embodiment, the compound administered is 3-[2,4-Diethyl-5-(2-oxo-5-phenylsulfamoyl-1,2-dihydro-indol-3-ylidenemethyl)-1H-pyrrol-3-yl]-propionic acid.

The invention also relates to an assay for determining the inhibition of DNA-PK kinase comprising the steps of:

(a) contacting purified DNA-PK with a peptide substrate inhibitor, double stranded activating DNA and appropriate buffer for a sufficient period of time to allow phosphorylation of the substrate;

(b) measuring substrate phosphorylation

In one aspect of the inventive assay, the double stranded activating DNA consists of an oligonucleotide annealed from oligonucleotides with the sequence of:

5′-TTTTTGGCCGCACGCGTCCACCATGGGGTACAACTACTTTTT-3′
and
5′-TTTTTGTAGTTGTACCCCATGGTGGACGCGTGCGGCCTTTTT-3′.

In another aspect of the inventive assay, the sequence of the peptide substrate is EPPLSQEAFADLWKK.

In another aspect of the inventive assay, the sequence of the peptide substrate is biotin-X-PESQEAFADLWKK.

In yet another aspect of the inventive assay, the sequence of the peptide substrate is SQEAFADLWKK.

Another aspect of the inventive assay involves detection of substrate phosphorylation by scintillation proximity assay.

Another aspect of the inventive assay relates to detection of substrate phosphorylation capture on a filter and scintillation counting.

In one aspect of the method of inhibiting DNA-PK, the patient undergoing such treatment has cancer.

In another aspect of the method of inhibiting DNA-PK, the cancer is selected from the group consisting of squamous cell carcinoma, astrocytoma, Kaposi's sarcoma, glioblastoma, lung cancer, bladder cancer, head and neck cancer, melanoma, ovarian cancer, prostate cancer, breast cancer, small-cell lung cancer, glioma, colorectal cancer, genitourinary cancer and gastrointestinal cancer.

### DETAILED DESCRIPTION OF THE PREFERRED EMBODIMENTS

Unless otherwise stated the following terms used in the specification and claims have the meanings discussed below:

“5-sulfonamido-indolinone” refers to a molecule having the chemical structure:

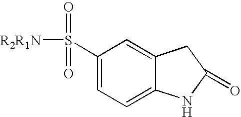

“Pharmaceutically acceptable salt” or “pharmaceutically acceptable salt thereof” refers to those salts which retain the biological effectiveness and properties of the free bases and which are obtained by reaction with inorganic or organic acids, such as hydrochloric acid, hydrobromic acid, hydroiodic acid, sulfuric acid, nitric acid, phosphoric acid, methanesulfonic acid, ethanesulfonic acid, p-toluenesulfonic acid, salicylic acid, acetic acid, benzenesulfonic acid (besylate), benzoic acid, camphorsulfonic acid, citric acid, fumaric acid, gluconic acid, glutamic acid, isethionic acid, lactic acid, maleic acid, malic acid, mandelic acid, mucic acid, pamoic acid, pantothenic acid, succinic acid, tartaric acid, and the like.

A “pharmaceutical composition” refers to a mixture of one or more of the compounds described herein, or pharmaceutically acceptable salts thereof, with other chemical components, such as pharmaceutically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives (including microcrystalline cellulose), gelatin, vegetable oils, polyethylene glycols, diluents, granulating agents, lubricants, binders, disintegrating agents, and the like.

“Alkyl” refers to a saturated aliphatic hydrocarbon including straight chain, branched chain or cyclic groups. Preferably, the alkyl group has 1 to 20 carbon atoms (whenever a numerical range; e.g., “1-20”, is stated herein, it means that the group, in this case the alkyl group, may contain 1 carbon atom, 2 carbon atoms, 3 carbon atoms, etc. up to and including 20 carbon atoms). More preferably, it is a medium size alkyl having 1 to 10 carbon atoms. Most preferably, it is a lower alkyl having 1 to 4 carbon atoms. The alkyl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

“Alkenyl” refers to an aliphatic hydrocarbon having at least one carbon-carbon double bond, including straight chain, branched chain or cyclic groups having at least one carbon-carbon double bond. Preferably, the alkenyl group has 2 to 20 carbon atoms (whenever a numerical range; e.g., “2-20”, is stated herein, it means that the group, in this case the alkenyl group, may contain 2 carbon atoms, 3 carbon atoms, etc. up to and including 20 carbon atoms). More preferably, it is a medium size alkenyl having 2 to 10 carbon atoms. Most preferably, it is a lower alkenyl having 2 to 6 carbon atoms. The alkenyl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

“Alkynyl” refers to an aliphatic hydrocarbon having at least one carbon-carbon triple bond, including straight chain, branched chain or cyclic groups having at least one carbon-carbon triple bond. Preferably, the alkynyl group has 2 to 20 carbon atoms (whenever a numerical range; e.g., “2-20”, is stated herein, it means that the group, in this case the alkynyl group, may contain 2 carbon atoms, 3 carbon atoms, etc. up to and including 20 carbon atoms). More preferably, it is a medium size alkynyl having 2 to 10 carbon atoms. Most preferably, it is a lower alkynyl having 2 to 6 carbon atoms. The alkynyl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

A “cycloalkyl” group refers to an all-carbon monocyclic or fused ring (i.e., rings which share an adjacent pair of carbon atoms) group wherein one of more of the rings does not have a completely conjugated pi-electron system. Examples, without limitation, of cycloalkyl groups are cyclopropane, cyclobutane, cyclopentane, cyclopentene, cyclohexane, adamantane, cyclohexadiene, cycloheptane and, cycloheptatriene. A cycloalkyl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

An “aryl” group refers to an all-carbon monocyclic or fused-ring polycyclic (i.e., rings which share adjacent pairs of carbon atoms) groups having a completely conjugated pi-electron system. Examples, without limitation, of aryl groups are phenyl, naphthalenyl and anthracenyl. The aryl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

An “aralkyl” group refers to an aryl group bonded to an alkyl moiety. Examples, without limitation, include benzyl, styryl and ethylbenzene. The aralkyl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

As used herein, a “heteroaryl” group refers to a monocyclic or fused ring (i.e., rings which share an adjacent pair of atoms) group having in the ring(s) one or more atoms selected from the group consisting of nitrogen, oxygen and sulfur and, in addition, having a completely conjugated pi-electron system. Examples, without limitation, of heteroaryl groups are pyrrole, furan, thiophene, imidazole, oxazole, thiazole, pyrazole, pyridine, pyrimidine, quinoline, isoquinoline, purine and carbazole. The heteroaryl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

A “heteroalicyclic ring” or “heteroalicycle” group refers to a monocyclic or fused ring group having in the ring(s) one or more atoms selected from the group consisting of nitrogen, oxygen and sulfur. The rings may also have one or more double bonds. However, the rings may not have a completely conjugated pi-electron system. The heteroalicyclic ring may be substituted or unsubstituted. The heteroalicyclic ring may contain one or more oxo groups. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

A “heteroalicycloalkyl” group refers to a heteroalicyclic ring bonded to an alkyl moiety. Examples include without limitation —CH₂-morpholinyl, —CH₂—CH₂-pyrrolidinyl and —CH₂-piperazinyl. The heteroalicycloalkyl group may be substituted or unsubstituted. When substituted, each substituent group is preferably one or more individually selected from halogen, -hydroxy, —COR′, —COOR′, OCOR′, —CONRR′, —RNCOR′, —NRR′, —CN, —NO₂, —CX₃, —SR′, —SOR′, —SO₂R′, —SO₂OR′, —SO₂NRR′, thiocarbonyl, —RNSO₂R′, perfluoroalkyl, O-carbamyl, N-carbamyl, O-thiocarbamyl, N-thiocarbamyl, silyl, ammonium, lower alkyl, lower alkenyl, lower alkynyl, cycloalkyl, heteroalicycle, heteroaryl and aryl. R and R′ are independently H, alkyl, or aryl, wherein alkyl or aryl may be further substituted with halogen, (CH₂)ₙN(R″)₂, (CH₂)ₙCO₂R″, (CH₂)ₙOR″, (CH₂)ₙOC(O)R″, alkoxycarbonyl, aryloxycarbonyl, aminocarbonyl, a heteroalicyclic ring, aryl, alkoxy, —OCX₃, aryloxy, C(O)NH₂ or heteroaryl. R″ is H, alkyl or aryl and n is 0-3.

X refers to a halogen group selected from the group consisting of fluorine, chlorine, bromine and iodine.

A “hydroxy” group refers to an —OH group.

A “protected hydroxy” group refers to a —OR, where the R is a protecting group, with a diol substituted compound a single protecting group can be used to form, e.g., a dioxolane (e.g., —O—CH2CH2-O—).

An “alkoxy” group refers to both an —O-alkyl and an —O-cycloalkyl group, as defined herein.

An “alkoxycarbonyl” refers to a —C(O)—OR.

An “aminocarbonyl” refers to a —C(O)—NRR′.

An “aryloxycarbonyl” refers to —C(O)-Oaryl.

An “aryloxy” group refers to both an —O-aryl and an —O-heteroaryl group, as defined herein.

An “arylalkyl” group refers to -alkyl-aryl, where alkyl and aryl are defined herein.

An “arylsulfonyl” group refers to a —S(O)n-aryl, wherein n is 0-2.

An “alkylsulfonyl” group refer to a —S(O)n-alkyl, wherein n is 0-2.

A “heteroaryloxyl” group refers to a heteroaryl —O— group with heteroaryl as defined herein.

A “heteroalicycloxy” group refers to a heteroalicyclic-O— group with heteroalicyclic as defined herein.

A “carbonyl” group refers to a —C(═O).

An “aldehyde” group refers to —C(═O)—R group where R is hydrogen.

A “thiocarbonyl” group refers to a —C(═S)—R group.

A “trihalomethanecarbonyl” group refers to a X₃C—C(O)— group.

A “C-carboxyl” group refers to a —C(O)O—R groups.

An “O-carboxyl” group refers to a R—C(O)O— group.

A “carboxylic acid” group refers to a C-carboxyl group in which R is hydrogen.

A “halo” or “halogen” group refers to fluorine, chlorine, bromine or iodine.

A “trihalomethyl” or a “trihaloalkyl” group refers to a —(CX₂)ₙ—CX₃ group, where n is 0 or greater.

A “trihalomethanesulfonyl” group refers to a X₃CS(O)₂ group.

A “trihalomethanesulfonamido” group refers to a X₃CS(O)₂NR— group.

A “sulfinyl” group refers to a —S(O)—R group.

A “sulfonyl” group refers to a —S(O)₂R group.

An “S-sulfonamido” group refers to a —S(O)₂NRR′ group.

An “N-Sulfonamido” group refers to a —NR—S(O)₂R group.

An “O-carbamyl” group refers to a —OC(O)NRR′ group.

An “N-carbamyl” group refers to a ROC(O)NRR′ group.

An “O-thiocarbarnyl” group refers to a —OC(S)NRR′ group.

An “N-thiocarbamyl” group refers to a ROC(S)NR′— group.

An “amino” group refers to an —NH₂ or an —NRR′ group.

A “C-amido” group refers to a —C(O)NRR′ group.

An “N-amido” group refers to a R′C(O)NR— group.

A “nitro” group refers to a —NO₂ group.

A “cyano” group refers to a —CN group.

A “silyl” group refers to a —Si(R)₃ group.

A “phosphonyl” group refers to a P(═O)(OR)₂ group.

A “polar” group refers to a group wherein the nuclei of the atoms covalently bound to each other to form the group do not share the electrons of the covalent bond(s) joining them equally; that is the electron cloud is denser about one atom than another. This results in one end of the covalent bond(s) being relatively negative and the other end relatively positive; i.e., there is a negative pole and a positive pole. Examples of polar groups include, without limitation, hydroxy, alkoxy, carboxy, nitro, cyano, amino, ammonium, amido, ureido, sulfonamido, sulfinyl, sulfonyl, phosphono, morpholino, piperazinyl and tetrazolo.

Compounds that have the same molecular formula but differ in the nature or sequence of bonding of their atoms or the arrangement of their atoms in space are termed “isomers”. Isomers that differ in the arrangement of their atoms in space are termed “stereoisomers.”

Stereoisomers that are not mirror images of one another are termed “diastereomers” and those that are non-superimposable mirror images of each other are termed “enantiomers”. When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R)- or (S)-stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are well-known in the art (see discussion in Chapter 4 of “Advanced Organic Chemistry”, 4th edition J. March, John Wiley and Sons, New York, 1992).

The compounds of Formula (I)-(VI) may exhibit the phenomena of tautomerism and structural isomerism. For example, the compounds described herein may adopt an E or a Z configuration about any double bond in the molecule. This invention encompasses any tautomeric or structural isomeric form and mixtures thereof which possess the ability to modulate DNA-PK activity and is not limited to any one tautomeric or structural isomeric form.

A “pharmaceutical composition” refers to a mixture of one or more of the compounds described herein, or physiologically/pharmaceutically acceptable salts or prodrugs thereof, with other chemical components, such as physiologically/pharmaceutically acceptable carriers and excipients. The purpose of a pharmaceutical composition is to facilitate administration of a compound to an organism.

The compound of Formula (I)-(VI) may also act as a prodrug. A “prodrug” refers to an agent which is converted into the parent drug in vivo. Prodrugs are often useful because, in some situations, they may be easier to administer than the parent drug. They may, for instance, be bioavailable by oral administration whereas the parent drug is not. The prodrug may also have improved solubility in pharmaceutical compositions over the parent drug. An example, without limitation, of a prodrug would be a compound of the present invention which is administered as an ester (the “prodrug”) to facilitate transmittal across a cell membrane where water solubility is detrimental to mobility but then is metabolically hydrolyzed to the carboxylic acid, the active entity, once inside the cell where water solubility is beneficial.

A further example of a prodrug might be a short polypeptide, for example, without limitation, a 2-10 amino acid polypeptide, bonded through a terminal amino group to a carboxy group of a compound of this invention wherein the polypeptide is hydrolyzed or metabolized in vivo to release the active molecule. The prodrugs of a compound of Formula (I)-(VI) are within the scope of this invention.

Additionally, it is contemplated that a compound of Formula (I)-(VI) would be metabolized by enzymes in the body of the organism such as a human being to generate a metabolite that can modulate the activity of DNA-PK. Such metabolites are within the scope of the present invention.

A general definition of “pharmaceutically acceptable salt” is set forth above. Furthermore, the term “pharmaceutically acceptable salt” refers to those salts which retain the biological effectiveness and properties of the parent compound. Such salts include:

(1) acid addition salt which is obtained by reaction of the free base of the parent compound with inorganic acids such as hydrochloric acid, hydrobromic acid, nitric acid, phosphoric acid, sulfuric acid, and perchloric acid and the like, or with organic acids such as acetic acid, oxalic acid, (D) or (L) malic acid, maleic acid, methanesulfonic acid, ethanesulfonic acid, p-toluenesulfonic acid, salicylic acid, tartaric acid, citric acid, succinic acid or malonic acid and the like, preferably hydrochloric acid or (L)-malic; or

(2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“PK” refers to receptor protein tyrosine kinase (RTKs), non-receptor or “cellular” tyrosine kinase (CTKs) and serine-threonine kinases (STKs).

“Method” refers to manners, means, techniques and procedures for accomplishing a given task including, but not limited to, those manners, means, techniques and procedures either known to, or readily developed from known manners, means, techniques and procedures by, practitioners of the chemical, pharmaceutical, biological, biochemical and medical arts.

“Modulation” or “modulating” refers to the alteration of the catalytic activity of DNA-PK.

“Catalytic activity” refers to the rate of phosphorylation of tyrosine under the influence, direct or indirect, of RTKs and/or CTKs or the phosphorylation of serine and threonine under the influence, direct or indirect, of STKs.

“Contacting” refers to bringing a compound of this invention and DNA-PK together in such a manner that the compound can affect the catalytic activity of the DNA-PK, either directly, i.e., by interacting with the kinase itself, or indirectly, i.e., by interacting with another molecule on which the catalytic activity of the kinase is dependent. Such “contacting” can be accomplished “in vitro,” i.e., in a test tube, a petri dish or the like. Cells may also be maintained or grown in cell culture dishes and contacted with a compound in that environment. In this context, the ability of a particular compound to affect DNA-PK, i.e., the IC₅₀ of the compound, defined below, can be determined before use of the compounds in vivo with more complex living organisms is attempted. For cells outside the organism, multiple methods exist, and are well-known to those skilled in the art, to contact the compounds with DNA-PK which include, but not limited to, direct cell microinjection and numerous transmembrane carrier techniques.

“In vitro” refers to procedures performed in an artificial environment such as, e.g., without limitation, in a test tube or culture medium.

“In vivo” refers to procedures performed within a living organism such as, without limitation, a mouse, rat or rabbit.

“Treat”, “treating” and “treatment” refer to a method of alleviating or abrogating a DNA-PK mediated disorder and/or its attendant symptoms. Here, a preferred method of “treatment” involves radiosensitization.

“Radiosensitizer” refer to agents which allow for modulation of the radiation dose required to obtain tumor control and limit the side effects without compromising treatment efficacy.

“Radiosensitization,” and “radiosensitize” refers to a method of making cell, tissues and organisms more susceptible to ionizing radiation.

“Organism” refers to any living entity comprised of at least one cell. A living organism can be as simple as, for example, a single eukaryotic cell or as complex as a mammal, including a human being.

“Therapeutically effective amount” refers to that amount of the compound being administered which will relieve to some extent one or more of the symptoms of the disorder being treated. In reference to the treatment of cancer, a therapeutically effective amount refers to that amount which has the effect of:

(1) reducing the size of the tumor;

(2) inhibiting (that is, slowing to some extent, preferably stopping) tumor metastasis;

(3) inhibiting to some extent (that is, slowing to some extent, preferably stopping) tumor growth, and/or,

(4) relieving to some extent (or, preferably, eliminating) one or more symptoms associated with the cancer.

“Cell phenotype” refers to the outward appearance of a cell or tissue or the biological function of the cell or tissue. Examples, without limitation, of a cell phenotype are cell size, cell growth, cell proliferation, cell differentiation, cell survival, apoptosis, and nutrient uptake and use. Such phenotypic characteristics are measurable by techniques well-known in the art.

“Natural binding partner” refers to a polynucleotide that binds to DNA-PK in a cell. A change in the interaction of the natural binding partner with DNA-PK can manifest itself as an increased or decreased concentration of DNA-PK/natural binding partner complex.

While not being bound to any particular theory, applicants at this time believe that the polar groups in the compounds of the invention may interact electronically, for example, but without limitation, through hydrogen bonds, Van der Walls forces and/or ionic bonds (but not covalent bonding), with one or more amino acids in DNA-PK by interfering with ATP binding. These interactions may assist the molecules of this invention to bind to an active site in DNA-PK with sufficient tenacity to interfere with or prevent the natural substrate from entering the site.

### EXAMPLES

### SYNTHESIS EXAMPLES

General Synthetic Scheme for Preparation of Amidosulfonyl Substituted Indolinones

Compounds of Formulae (I)-VI) may be prepared by methods described in U.S. Pat. Nos. 6,130,238; 6,395,734 and 6,465,507 which are hereby incorporated by reference; and U.S. patent application Ser. No. 09/129,256, filed Aug. 4, 1998; Ser. No. 09/783,263, filed Feb. 15, 2001; Ser. No. 09/871,700, filed Jun. 4, 2001; Ser. No. 10/130,978, filed May 24, 2002; Ser. No. 10/157,007, filed May 30, 2002 and Ser. No. 10/238,051, filed Sep. 10, 2002, was of which is hereby incorporated by reference.

The following general methodology also may be employed to prepare the compounds of this invention.

Amidosulfonyl-indolinones were synthesized by condensation of an appropriately substituted oxindole (0.9 equivalent), an appropriately substituted pyrrole aldehyde (1 equivalent) and piperidine (excess) in ethanol (0.2 M) between room temperature and 100° C. as shown in the following scheme.

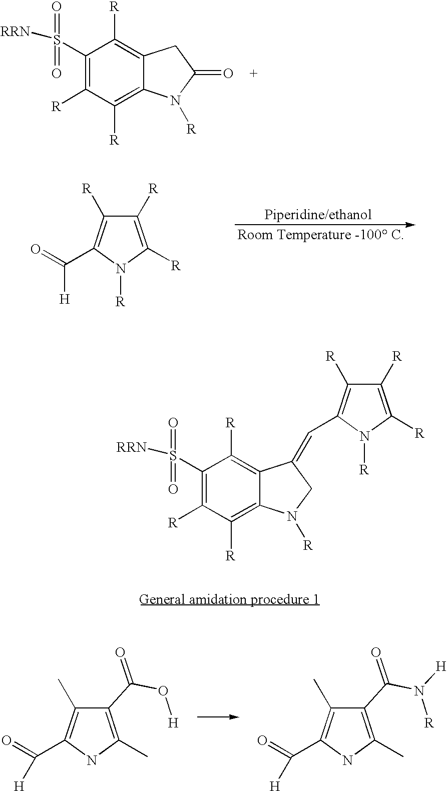

5-Formyl-2,4-dimethyl-1H-pyrrole-3-carboxylic acid (1 equivalent) is dissolved in dimethylformamide (0.3 M) with stirring. To this is added 1-ethyl-3-(3-dimethylamino-propylcarbodiimide hydrochloride, 1.2 equivalents), 1-hydroxybenzotriazole (1.2 equivalents) followed by triethylamine (2 equivalents). The appropriate amine is added (1 equivalent) and the reaction stirred for 12 hours. The reaction is diluted with saturated sodium bicarbonate, sodium hydroxide solution, brine and additional solid sodium chloride and extracted twice with 10% methanol in dichloromethane. The combined organic layers are washed with brine, dried over anhydrous magnesium sulfate and concentrated. The resulting oil is re-concentrated from toluene and precipitated from diethyl ether/hexanes to afford a solid.

1. Aldehydes

A-1 5-Formyl-2,4-dimethyl-1H-pyrrole-3-carboxylic acid

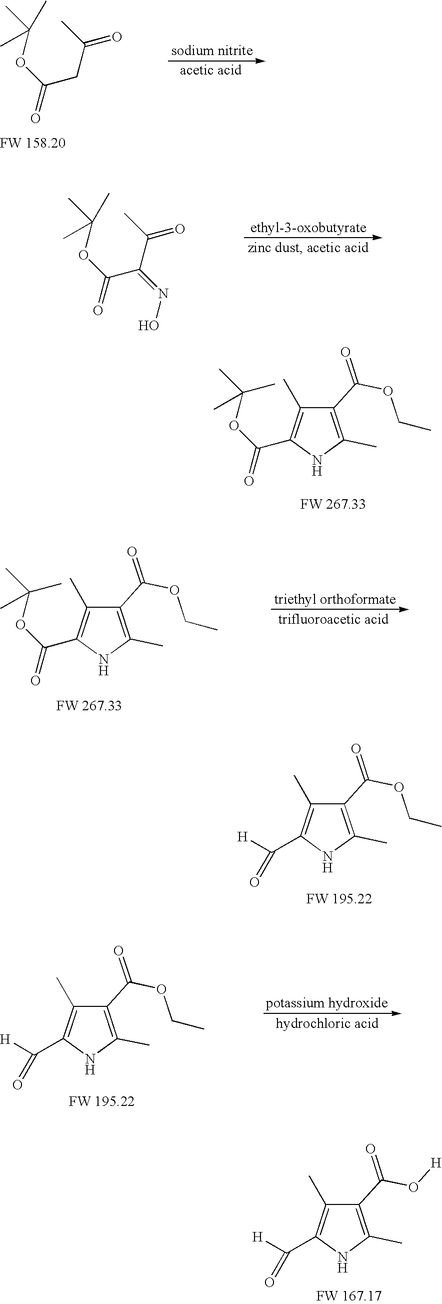

t-Butyl-3-oxobutyrate (158 g, 1 mol) was dissolved in 200 mL of acetic acid in a 500 mL 3-neck round bottom flask equipped with a thermometer, an addition funnel and mechanical stirring. The mixture was cooled in an ice bath to about 10° C. Sodium nitrite (69 g, 1 mol) was added over 75 minutes keeping the temperature under 15° C. The cold bath was removed and the mixture stirred for 30 minutes and then allowed to stand for 3.5 hours to give a solution of crude t-butyl-2-hydroximino-3-oxobutyrate.

Ethyl-3-oxobutyrate (130 g, 1 mol) was dissolved in 400 mL of acetic acid in a 2 L 3-neck round bottom flask equipped with a thermometer, an addition funnel and mechanical stirring and placed in an oil bath. Zinc dust (50 g, 0.76 mol) was added and the mixture heated to 60° C. with stirring. The crude t-butyl-2-hydroximino-3-oxobutyrate solution prepared above was cautiously added keeping the temperature at about 65° C. by slowing the addition and cooling the flask. More zinc dust (4×50 g, 3.06 mol) was added in portions during the addition with the last portion added after all the t-butyl ester had been added. The temperature of the mixture reached a maximum of 80° C. At the end of the additions the temperature was 64° C. The temperature was increased by heating to 70-75° C. for one hour and then poured into 5 L of water. The gray floating precipitate was collected by vacuum filtration and washed with 2 L of water to give 354 g of wet crude product. The crude product was dissolved in 1 L of hot methanol and hot filtered to remove zinc. The filtrate was cooled to give a precipitate that was collected by vacuum filtration to give 118 g of product. The filtrate was put in the refrigerator overnight to give a total of 173.2 g of 3,5-dimethyl-1H-pyrrole-2,4-dicarboxylic acid 2-tert-butyl ester 4-ethyl ester as an off-white solid.

3,5-Dimethyl-1H-pyrrole-2,4-dicarboxylic acid 2-tert-butyl ester 4-ethyl ester (80.1 g, 0.3 mol) and 400 mL of trifluoroacetic acid were stirred for 5 minutes in a 2 L 3-neck round bottom flask equipped with mechanical stirring and warmed to 40° C. in an oil bath. The mixture was then cooled to −5° C. and triethyl orthoformate (67.0 g, 0.45 mol) was added all at once. The temperature increased to 15° C. The mixture was stirred for about 1 minute, removed from the cold bath and then stirred for 1 hour. The trifluoroacetic acid was removed by rotary evaporation and the residue put in the refrigerator where it solidified. The solid was dissolved by warming and poured into 500 g of ice. The mixture was extracted with 800 mL of dichloromethane to give a red solution and a brown precipitate, both of which were saved. The precipitate was isolated and washed with 150 mL of saturated sodium bicarbonate solution. The dichloromethane phase was washed with 150 mL of sodium bicarbonate and both bicarbonate solutions discarded. The dichloromethane solution was washed with 3 times with 100 mL of water each time. The dichloromethane solution was evaporated to dryness and the dark residue recrystallized twice from hot ethyl acetate after decolorizing with Darco to give golden yellow needles. The brown precipitate was recrystallized from 350 mL of hot ethyl acetate after decolorizing with Darco to give a yellow-red solid. All the recrystallized solids were combined and recrystallized from 500 mL of hot ethanol to give 37.4 g (63.9%) of 5-formyl-2,4-dimethyl-1H-pyrrole-3-carboxylic acid ethyl ester as yellow needles (mp 165.6-166.3° C., lit. 163-164° C.). The evaporated residues from the ethyl acetate and ethanol mother liquors were recrystallized from 500 mL of ethanol to give 10.1 g (17.1%) of a second crop of dirty yellow needles.

5-Formyl-2,4-dimethyl-1H-pyrrole-3-carboxylic acid ethyl ester (2 g, 10 mmol) was added to a solution of potassium hydroxide (3 g, 53 mmol) dissolved in methanol (3 mL) and water (10 mL). The mixture was refluxed for 3 hours, cooled to room temperature and acidified with 6 N hydrochloric acid to pH 3. The solid was collected by filtration, washed with water and dried in a vacuum oven overnight to give 1.6 g (93%) of 5-formyl-2,4-dimethyl-1H-pyrrole-3-carboxylic acid. ¹H NMR (300 MHz, DMSO-d6) δ 12.09 (s, br, 2H, NH & COOH), 9.59 (s, 1H, CHO), 2.44 (s, 3H, CH₃), 2.40 (s, 3H, CH₃).

A-2 3,5-Dimethyl-4-(piperazine-1-carbonyl)-1H-pyrrole-2-carbaldehyde

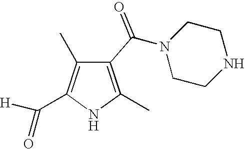

MS m/z 236 [M+1].

A-3 4-(3,5-Dimethyl-piperazine-1-carbonyl)-3,5-dimethyl-1H-pyrrole-2-carbaldehyde

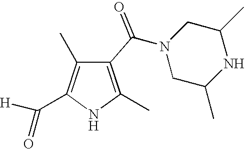

5-Formyl-2,4-dimethyl-1H-pyrrole-3-carboxylic acid (2 g, 11.96 mmol) was reacted with cis-2,6-dimethylpiperazine (2.66 g, 14.36 mmol) to give 2.27 g (72% yield) of 3,5-dimethyl-4-[(3R,5S)-3,5-dimethyl-piperazine-1-carbonyl]-1H-pyrrole-2-carbaldehyde using General Amidation Procedure 1. ¹H-NMR (360 MHz, diemthylsulfoxide-d6) δ 11.84 (br s, 1H, NH), 9.51 (s, 1H, CHO), 4.30 (br s 1H, NH), 2.50 (m, 4H, 2×CH₂), 2.28 (m, 8H, 2×CH₃ and 2×CH), 0.96 (m, 6H, 2×CH₃). MS m/z 264 [M+1].

A-4 4-[4-(2-Hydroxy-ethyl)-piperazine-1-carbonyl]-3,5-dimethyl-1H-pyrrole-2-carbaldehyde

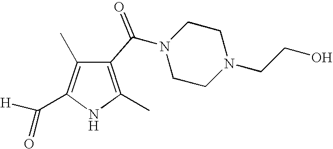

MS m/z 280 [M+1].

A-5 5-Formyl-2,4-dimethyl-1H-pyrrole-3-carboxylic acid [3-(4-methyl-piperazin-1-yl)-propyl]-amide

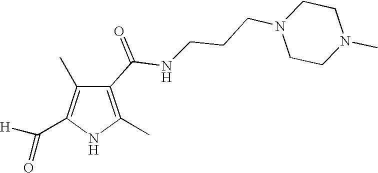

5-Formyl-2,4-dimethyl-1H-pyrrole-3-carboxylic acid (1.0 g, 6 mmol) was reacted with 1-(3-aminopropyl)-4-methylpiperazine (1.06 mL) to give 0.38 g (23% yield) of 5-formyl-2,4-dimethyl-1H-pyrrole-3-carboxylic acid [3-(4-methyl-piperazin-1-yl)-propyl]-amide using General Amidation Procedure 1. ¹H-NMR (360 MHz, dimethylsulfoxide-d6) δ 11.8 (br s, 1H, NH), 9.52 (s, 1H, CHO), 7.52 (m, 1H, CONH), 3.2 (m, 2H, CH₂), 2.8 (m, 2H, CH₂), 2.5 (m, 8H, 4×CH₂), 2.34 (s, 3H, CH₃), 2.29 (s, 3H, CH₃), 1.68 (m, 2H, CH₂).

MS m/z 307 [M+1].

A-6 5-Formyl-1H-pyrrole-2-carboxylic acid

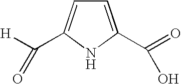

To a solution of dimethylformamide (21 mL, 0.27 mol) in 75 mL of dichloroethane was added a solution of phosphorus oxychloride (25 mL, 0.27 mol) in 75 mL of dichloroethane at 0° C. The mixture was stirred at room temperature for 30 minutes and cooled to 0° C. To the mixture was added a solution of ethyl pyrrole-2-carboxylate (25 g, 0.18 mol) in 50 mL of dichloroethane dropwise. The resulting mixture was stirred at room temperature for 30 minutes and then at 40° C. for 1 hour. The mixture was poured into ice and basified to pH 11 with 5 N sodium hydroxide solution. The mixture was extracted with ethyl acetate and the extract washed with water and brine and dried over anhydrous sodium sulfate. The two products were separated by column chromatography (1:3 ethyl acetate:hexane) to give 15 g (50% yield) of 5-formyl-1H-pyrrole-2-carboxylic acid ethyl ester and 2 g (7% yield) of 4-formyl-1H-pyrrole-2-carboxylic acid ethyl ester. ¹H-NMR (300 MHz, dimethylsulfoxide) δ 13.02 (br s, 1H, NH), 9.69 (s, 1H, CHO), 6.95 (d, 1H), 6.86 (d, 1H), 4.27 (q, 2H, CH₃), 1.28 (t, 3H, CH₃). MS M/Z 167 [M+].

Refluxing 4-formyl-1H-pyrrole-2-carboxylic acid ethyl ester in 2 equivalents of potassium hydroxide in methanol:water followed by acidification to pH 3 at 0° C. gave 4-formyl-1H-pyrrole-2-carboxylic acid as a solid. MS m/z 140 [M+1].

A-7 3,5-Dimethyl-4-(4-methyl-piperazine-1-carbonyl)-1H-pyrrole-2-carbaldehyde

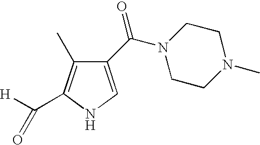

MS m/z 250 [M+1].

A-8 3,5-Dimethyl-4-(3-morpholin-4-yl-propyl)-1H-pyrrole-2-carbaldehyde

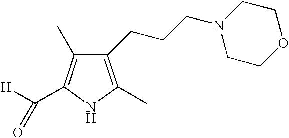

4-(2-Methoxycarbonyl-ethyl)-3,5-dimethyl-1H-pyrrole-2-carboxylic acid ethyl ester (Aldrich, 228 g, 0.9 mol) and 720 mL of 5 N sodium hydroxide were refluxed for one hour. Thin layer chromatography showed the hydrolysis to be complete. The stirred mixture was cooled to 50° C. and hydrochloric acid (10 N, 390 mL) was slowly added to give a pH of 2-3. The oil which separated solidified. The mixture was stirred and cooled to 4° C. The solids were collected by vacuum filtration, washed thoroughly with water and dried under vacuum at 40° C. to give 65.7 g (49% yield) of 3-(2,4-dimethyl-1H-pyrrol-3-yl)-propionic acid as a reddish solid.

To a suspension of 10 g (60.8 mmol) of 3-(2,4-dimethyl-1H-pyrrol-3-yl)-propionic acid in 60 mL of dichloromethane was added 11.6 gm (71.8 mmol) of 1,1′-carbonyldiimidazole followed by 5.5 mL (60.8 mmol) of morpholine and 10 mL (60.8 mmol) of N,N-diisopropylethylamine. The dark red reaction mixture was stirred at room temperature overnight and poured into ice water. The organic layer was washed with brine until its pH was 6, dried over anhydrous sodium sulfate, and concentrated. The crude product was purified on a silica gel column eluting with dichloromethane-methanol (98:2) to give 13.84 g (96% yield) of 3-(2,4-dimethyl-1H-pyrrol-3-yl)-1-morpholin-4-yl-propan-1-one.

To a suspension of 2.67 g (70 mmol) of lithium aluminum hydride in 100 mL of tetrahydrofuran was added dropwise a solution of 13.84 g (59 mmol) of 3-(2,4-dimethyl-1H-pyrrol-3-yl)-1-morpholin-4-yl-propan-1-one in 50 mL of tetrahydrofuran. The reaction mixture was stirred at 80° C. for 1 hour and cooled in an ice bath. Ice cubes were slowly added to the mixture until no more gas was generated. A few drops of 2 N sodium hydroxide were added and the reaction mixture was stirred at room temperature for 30 min, extracted with ethyl acetate and the extract dried over anhydrous sodium sulfate and concentrated to give 10.37 g (79% yield) of 4-[3-(2,4-dimethyl-1H-pyrrol-3-yl)-propyl]-morpholine as a light brown oil which was used without further purification.

To an ice-cooled solution of 5.5 mL (70 mmol) of dimethylformamide in 30 mL of dichloromethane was added 6.5 mL (70 mmol) of phosphorus oxychloride dropwise. The reaction mixture was stirred at room temperature for 15 minutes and a solution of 10.37 g (46.6 mmol) of 3,5-dimethyl-4-(3-morpholin-4-yl-propyl)-1H-pyrrole-2-carbaldehyde in 20 mL of dichloromethane was added dropwise at 0° C. The mixture was refluxed at 60° C. for 4 hours and cooled in an ice bath. Ice cubes were slowly added to the mixture followed by addition of 2 N sodium hydroxide to adjust the pH to 12. The reaction mixture was stirred at room temperature for 30 minutes and extracted with ethyl acetate. The organic layer was washed with brine, dried over anhydrous sodium sulfate and concentrated to give crude product which was purified on a silica gel column eluting with dichloromethane-methanol-ammonium hydroxide (9.5:0.5) to give 4.57 g (39% yield) of 3,5-dimethyl-4-(3-morpholin-4-yl-propyl)-1H-pyrrole-2-carbaldehyde as a dark red oil. ¹H-NMR (360 MHz, dimethylsulfoxide-d6) δ 11.34 (brs, 1H, NH), 9.40 (s, 1H, CHO), 3.55 (t, 4H, 2×CH₂), 2.28-2.34 (m, 6H, 2×CH₂), 2.21 (t, 4H, 2×CH₂) 2.19 (s, 3H, CH₃), 2.14 (s, 3H, CH₃), 1.51 (quintet, 2H, CH₂). MS m/z 251 [M+1]+.

A-9 3-(2-Hydroxy-ethyl)-4-(4-methyl-piperazine-1-carbonyl)-1H-pyrrole-2-carbaldehyde

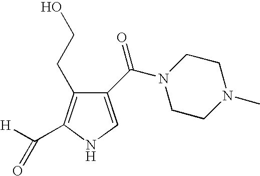

MS m/z 266 [M+1].

A-10 2-(5-Formyl-2,4-dimethyl-1H-pyrrol-3-yl)-N-(2-pyrrolidin-1-yl-ethyl)-acetamide

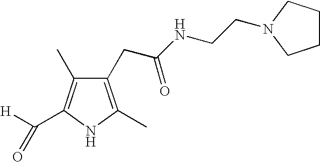

3-Acetyl-4-oxo-pentanoic acid ethyl ester (710 g, 3.81 mol), 533 mL of acetic acid and 847 g (4.00 mol) of diethyl aminomalonate hydrochloride salt were heated in an oil bath while 344 g (4.19 mol) of sodium acetate were slowly added. The internal temperature at the end of the addition was 55° C. and the mixture was thick with solids but could be stirred. The mixture was heated to 105° C. Gas evolution was vigorous. The temperature was allowed to fall to 98° C. and the reaction was stirred at this temperature for 30 minutes. Thin layer chromatography (ethyl acetate:hexane 1:2) showed the product at Rf 0.6 and a small impurity at Rf 0.7. The reaction was cooled to 50° C. and 1000 mL of ethanol and 1000 mL of water were added with stirring. The mixture was cooled in an ice bath and 3000 mL of ice water were added with stirring. The solids were collected by vacuum filtration and washed two times with 500 mL of 30% ethanol in water each time. The solids were air dried to give 870 g (93% yield) of 4-ethoxycarbonylmethyl-3,5-dimethyl-1H-pyrrole-2-carboxylic acid ethyl ester. ¹H-NMR (dimethylsulfoxide-d6) δ 1.15, 1.26 (2×t, 2×3H, 2×CH₃), 2.15 (2×s, 2×3H, 2×CH₃), 3.32 (s, 2H, CH₂), 4.01, 4.18 (2×d, 2×2H, 2×CH₂O), 11.20 (br s, 1H, NH). MS m/z 254 [M+1].

4-Ethoxycarbonylmethyl-3,5-dimethyl-1H-pyrrole-2-carboxylic acid ethyl ester (1000 g, 3.95 mol), 2600 mL of water, 300 mL of ethanol and 700 g (11.2 mol) of potassium hydroxide were refluxed at 88-92° C. for 55 minutes. Thin layer chromatography (ethyl acetate:hexane:acetic acid 4:6:0.5) showed a new spot at the origin and the absence of starting material. The mixture was cooled to 10° C. and 1200 mL of 10 N hydrochloric acid were added to a pH of 2.5. The maximum temperature reached was 14° C. The mixture was stirred in an ice bath for 1 hour. The solids were collected by vacuum filtration and washed twice with 200 mL of water each time. The solids were used wet in the decarboxylation step.

4-Carboxymethyl-3,5-dimethyl-1H-pyrrole-2-carboxylic acid wet mixture (778 g, 3.95 mol), 880 mL of 9 N potassium hydroxide and 880 mL of water were heated to an internal temperature of 65° C., removed from the heat, and 800 mL of 10 N hydrochloric acid were slowly added with stirring to a pH of 2.5. The temperature increased to 68° C. and then decreased to 58° C. with the evolution of carbon dioxide. Thin layer chromatography (ethyl acetate:hexane:acetic acid 4:6:0.5) showed a major spot at Rf 0.7 and a small spot at the origin. The mixture was cooled to 4° C. The solids were collected by vacuum filtration and washed 3 times with 200 mL of water each time. The solid was air dried to give 240 g (40% yield) of (2,4-dimethyl-1H-pyrrol-3-yl)-acetic acid as a brown solid. ¹H-NMR (dimethylsulfoxide-d6) δ 1.89, 2.06 (2×s, 2×3H, 2×CH₃), 3.17 (s, 2H, CH₂), 6.26 (s, 1H, pyrrole CH), 10.02 (s, 1H, NH), 11.74 (br s, 1H, COOH).

Dimethylformamide (230 mL, 3.13 mol) and 1200 mL of dichloromethane were cooled to 4° C. and 222 mL of phosphorus oxychloride was added with stirring. The temperature increased to 20° C. The mixture was cooled to 2° C. (2,4-Dimethyl-1H-pyrrol-3-yl)-acetic acid (240 g, 1.57 mol) was slowly added. The mixture was refluxed for 20 minutes. Thin layer chromatography (ethyl acetate:hexane:acetic acid 4:6:0.5) showed no starting material at Rf 0.7 and a heavy spot at the origin. The mixture was cooled to 5° C. and 1200 mL of ice water were added. The maximum temperature reached was 30° C. The mixture was stirred in an ice bath for 30 minutes. The dichloromethane layer was separated and discarded. The aqueous layer was cooled to 9° C. and adjusted to pH 12 with about 1650 mL of 9 N potassium hydroxide. The maximum temperature reached was 50° C. The mixture was cooled in an ice bath and adjusted to pH 3 with about 800 mL of 10 N hydrochloric acid. The solids were collected by vacuum filtration, washed three times with 300 mL of water each time and air dried to give 274 g (96% yield) of (5-formyl-2,4-dimethyl-1H-pyrrol-3-yl)-acetic acid as a brown solid. ¹H-NMR (dimethylsulfoxide-d6) δ 2.15, 2.18 (2×s, 2×3H, 2×CH₃), 3.28 (s, 2H, CH₂), 9.44 (s, 1H, CHO), 11.50 (s, 1H, pyrrole NH), 12.07 (s, 1H, COOH). MS m/z 182, [M+1].

(5-Formyl-2,4-dimethyl-1H-pyrrol-3-yl)-acetic acid was reacted with 1-(2-aminoethyl)pyrrolidine using General Amidation Procedure 1 to give 2-(5-formyl-2,4-dimethyl-1H-pyrrol-3-yl)-N-(2-pyrrolidin-1-yl-ethyl)-acetamide. MS m/z 278 [M+1].

A-11 5-Formyl-2,4-dimethyl-1H-pyrrole-3-carboxylic acid (2-diethylamino-ethyl)-amide

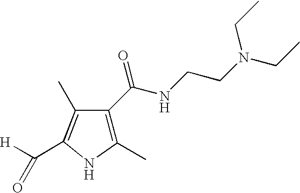

5-Formyl-2,4-dimethyl-1H-pyrrole-3-carboxylic acid (0.5 g, 2.99 mmol) was reacted with dimethylaminoethylamine (0.32 mL) to give 0.37 g (52% yield) of 5-formyl-2,4-dimethyl-1H-pyrrole-3-carboxylic acid (2-dimethylamino-ethyl)-amide by using General Amidation Procedure 1, ¹H-NMR (360 MHz, dimethylsulfoxide-d6) δ 11.84 (br s, 1H, NH), 9.52 (s, 1H, CHO), 7.47 (m, 1H, NH), 3.3 (m, 2H, CH₂), 2.55 (m, 2H, CH₂), 2.35 (s, 3H, CH₃), 2.29 (s, 6H, 2×CH₃), 2.30 (s, 3H, CH₃). MS m/z 266 [M+1].

A-12 N-(2-Diethylamino-ethyl)-2-(5-formyl-2,4-dimethyl-1H-pyrrol-3-yl)-acetamide

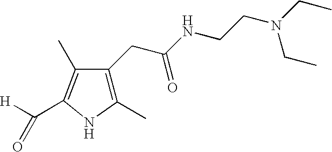

(5-Formyl-2,4-dimethyl-1H-pyrrol-3-yl)-acetic acid was reacted with diethylaminoethylamine using General Amidation Procedure 1 to give N-(2-diethylamino-ethyl)-2-(5-formyl-2,4-dimethyl-1H-pyrrol-3-yl)-acetamide. MS m/z 280 [M+1].

A-13 3-(3-Dimethylamino-propyl)-6,6-dimethyl-4,5,6,7-tetrahydro-1H-indole-2-carbaldehyde

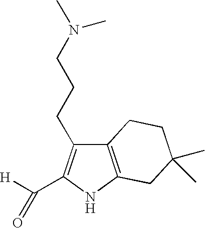

To a suspension of 3-(6,6-dimethyl-4-oxo-4,5,6,7-tetrahydro-1H-indol-3-yl)-propionic acid (1.65 g, 7 mmol) in dichloromethane (15 mL) was added 1.36 g (8.4 mmol) of carbonyldiimidazole. The mixture was stirred at room temperature until the solution became clear. To the mixture was added dimethylamine (14 mL of a 2.0 M solution in tetrahydrofuran). The mixture was stirred at room temperature overnight and then concentrated. The residue was redissloved in dichloromethane, washed with brine and concentrated. The solid was washed with ethyl acetate until it became white and dried to give 1.4 g (78% yield) of 3-(6,6-dimethyl-4-oxo-4,5,6,7-tetrahydro-1H-indol-3-yl)-N,N-dimethyl-propionamide. ¹H-NMR (360 MHz, diemthylsulfoxide-d6) δ10.93 (br s, 1H, NH), 6.48 (s, 1H), 2.94 (s, 3H, CH₃), 2.79 (s, 3H, CH₃), 2.72 (m, 2H, CH₂), 2.57 (s, 2H, CH₂), 2.48 (m, 2H, CH₂), 2.17 (s, 2H, CH₂), 1.00 (m, 6H, 2×CH₃). MS m/z 262 [M+].

To a suspension of 3-(6,6-dimethyl-4-oxo-4,5,6,7-tetrahydro-1H-indol-3-yl)-N,N-dimethyl-propionamide (1.2 g, 4.68 mmol) in tetrahydrofuran (30 mL) was added dropwise a solution of lithium aluminum hydride (0.69 g, 18.3 mmol) in tetrahydrofuran. The mixture was refluxed overnight. Ice was added to the cooled reaction until no more gas was generated followed by 15% sodium hydroxide and water. The reaction was stirred at room temperature for 30 minutes and the insolubles removed by vacuum filtration. The filtrate was concentrated to give [3-(6,6-dimethyl-4,5,6,7-tetrahydro-1H-indol-3-yl)-propyl]-dimethyl-amine as an oil. ¹H-NMR (360 MHz, dimethylsulfoxide-d6) δ 9.75 (br s, 1H, NH), 6.24 (s, 1H), 3.36 (m, 2H, CH₂), 2.07-2.3 (m, 14H, 7×CH₂), 1.53 (m, 2H, CH₂), 1.40 (m, 2H, CH₂), 0.92 (s, 6H, 2×CH₃). MS m/z 234 [M+].

Phosphorus oxychloride (0.5 mL, 5.1 mmol) was added dropwise to ice-cooled dimethylformamide (1.1 mL, 14.1 mmol) and then stirred at room temperature for 30 minutes. A solution of [3-(6,6-dimethyl-4,5,6,7-tetrahydro-1H-indol-3-yl)-propyl]-dimethyl-amine (1.1 g, 4.6 mmol) in dimethylformamide (2 mL) was added dropwise at −5° C. The mixture was stirred at room temperature overnight. Ice cubes were added to the reaction mixture followed by the addition of 10 N potassium hydroxide to pH 11-12 and stirring for one hour. The mixture was extracted with ethyl acetate and the extract washed with brine and concentrated. The residue was chromatographed (5%-10% methanol in dichloromethane) to give 650 mg of 3-(3-dimethylamino-propyl)-6,6-dimethyl-4,5,6,7-tetrahydro-1H-indole-2-carbaldehyde as a brown solid. ¹H-NMR (360 MHz, dimethylsulfoxide-d6) δ 11.25 (br s, 1H, NH), 9.41 (s, 1H, CHO), 2.62 (t, 2H, CH₂), 2.36 (t, 2H, CH₂), 2.30 (s, 2H, CH₂), 2.21 (t, 2H, CH₂), 2.12 (s, 6H, 2×CH₃), 1.60 (m, 2H, CH₂), 1.46 (t, 2H, CH₂), 0.93 (s, 6H, 2×CH₃). MS m/z 262 [M+].

A-14 2-(5-Formyl-2,4-dimethyl-1H-pyrrol-3-yl)-N-(2-morpholin-4-yl-ethyl)-acetamide

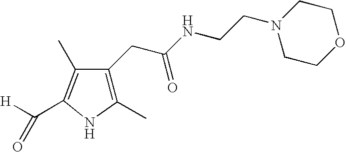

MS m/z 294 [M+1].

A-15 3-(3-Diethylamino-propyl)-6,6-dimethyl-4,5,6,7-tetrahydro-1H-indole-2-carbaldehyde

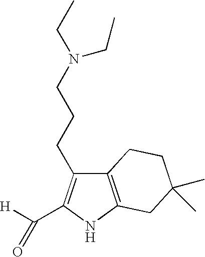

A mixture of 5-aminolevulinic acid hydrochloride (1.68 g, 10 mmol), 5,5-dimethyl-1,3-cyclohexandione (1.4 g, 10 mmol) and sodium acetate (1.64 g, 20 mmol) in water (10 mL) was heated to at 110° C. for 4 hours and then cooled. The resulting solid was collected by vacuum filtration, washed with 30% ethanol in water and dried under vacuum to give 1.6 g (68% yield) of 3-(6,6-dimethyl-4-oxo-4,5,6,7-tetrahydro-1H-indol-3-yl)-propionic acid. ¹H-NMR (360 MHz, dimethylsulfoxide-d6) δ 11.89 (br s, 1H, COOH), 10.94 (br s, 1H, NH), 6.45 (d, 1H), 2.76 (t, 2H, CH₂), 2.57 (s, 2H, CH₂), 2.44 (t, 2H, CH₂), 2.16 (s, 2H, CH₂), 0.99 (s, 6H, 2×CH₃). MS m/z 235 [M+].

To a suspension of 1.18 g of 3-(6,6-dimethyl-4-oxo-4,5,6,7-tetrahydro-1H-indol-3-yl)-propionic acid (5 mmol) in dichloromethane (25 mL) was added 0.97 g (6 mmol) of carbonyldiimidazole. After stirring at room temperature for 2 hours, 2.1 mL (20 mmol) of diethylamine was added. The mixture was stirred at room temperature overnight. The reaction was concentrated and the residue dissolved in dichloromethane, washed with brine, dried and concentrated to give 1.2 g (83% yield) of 3-(6,6-dimethyl-4-oxo-4,5,6,7-tetrahydro-1H-indol-3-yl)-N,N-diethyl-propionamide as a white solid. ¹H-NMR (360 MHz, dimethylsulfoxide-d6) δ 10.91 (br s, 1H, NH), 6.46 (s, 1H), 3.20-3.29 (m, 4H, 2×CH₂), 2.72-2.76 (m, 2H, CH₂), 2.57 (s, 2H, CH₂), 2.45 (m, 2H, CH₂), 2.17 (s, 2H, CH₂), 0.96-1.06 (m, 12H, 4×CH₃). MS m/z 290 [M+].

To a suspension of 3-(6,6-dimethyl-4-oxo-4,5,6,7-tetrahydro-1H-indol-3-yl)-N,N-diethyl-propionamide (1.1 g, 3.8 mmol) in tetrahydrofuran (80 mL) was added dropwise a solution of lithium aluminum hydride (0.57 g, 15.1 mmol) in tetrahydrofuran. The mixture was refluxed overnight. To the cooled reaction was added enough ice until no more gas was generated followed by 15% sodium hydroxide and water. The reaction was stirred at room temperature for 30 minutes and the insolubles removed by vacuum filtration. The filtrate was concentrated to give 0.9 g of [3-(6,6-dimethyl-4,5,6,7-tetrahydro-1H-indol-3-yl)-propyl]-diethyl-amine as a light yellow oil. ¹H-NMR (360 MHz, dimethylsulfoxide-d6) δ 9.75 (br s, 1H, NH), 6.24 (s, 1H), 2.19-2.44 (m, 14H, 7×CH₂), 1.53 (m, 2H, CH₂), 1.40 (m, 2H, CH₂), 0.88-0.92 (m, 12H, 4×CH₃). MS m/z 262 [M+].

Phosphorus oxychloride (0.35 mL, 3.74 mmol) was added dropwise to cooled dimethylformamide (0.8 mL, 10.3 mmol). After stirring at room temperature for 30 minutes, the mixture was cooled to −5° C. and a solution of [3-(6,6-dimethyl-4,5,6,7-tetrahydro-1H-indol-3-yl)-propyl]-diethyl-amine (0.9 g, 3.4 mmol) in dimethylformamide (2 mL) was added dropwise. The mixture was stirred at room temperature for 3 hours. The reaction was quenched with ice, followed by 10 N potassium to adjust the pH to 10-11. After stirring at room temperature for 1 hour, the reaction was extracted with ethyl acetate and the extract was washed with brine, dried and concentrated to give 0.55 g of 3-(3-diethylamino-propyl)-6,6-dimethyl-4,5,6,7-tetrahydro-1H-indole-2-carbaldehyde. ¹H-NMR (360 MHz, dimethylsulfoxide-d6) δ 11.23 (br, s, 1H, NH), 9.41 (s, 1H, CHO), 2.61 (t, 2H, CH₂), 2.30-2.43 (m, 10H, 5×CH₂), 1.58 (m, 2H, CH₂), 1.45 (t, 2H, CH₂), 0.93 (s, 6H, 2×CH₃), 0.89 (t, 6H, CH₃). MS m/z 290 [M+].

A-16 5-Formyl-2,4-dimethyl-1H-pyrrole-3-carboxylic acid (2-pyrrolidin-1-yl-ethyl)-amide

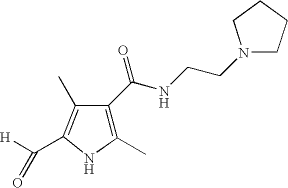

5-Formyl-2,4-dimethyl-1H-pyrrole-3-carboxylic acid (0.5 g, 2.99 mmol) was reacted with 1-(2-aminoethyl)pyrrolidine (0.42 mL) to give 0.57 g (73% yield) of 5-formyl-2,4-dimethyl-1H-pyrrole-3-carboxylic acid (2-pyrrolidin-1-yl-ethyl)-amide using General Amidation Procedure 1. ¹H-NMR (360 MHz, dimethylsulfoxide-d6) δ 11.79 (br s, 1H, NH), 9.53 (s, 1H, CHO), 7.41 (m, 1H, NH), 3.28-3.34 (m, 2H, CH₂), 2.53-2.60 (m, 6H, CH₂ and 2×CH₂), 2.35 (s, 3H, CH₃), 2.3 (s, 3H, CH₃), 1.68 (m, 4H, 2×CH₂). MS m/z 264 [M+1].

A-17 2-Ethyl-5-formyl-4-methyl-1H-pyrrole-3-carboxylic acid (2-pyrrolidin-1-yl-ethyl)-amide

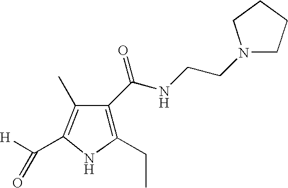

MS m/z 278 [M+1].

A-18 5-Formyl-2-methyl-4-[3-(4-methyl-piperazin-1-yl)-propyl]-1H-pyrrole-3-carboxylic acid ethyl ester

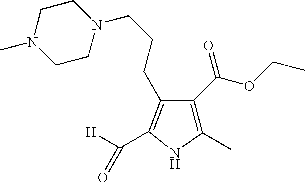

MS m/z 322 [M+1].

A-19 5-Formyl-2-methyl-4-(3-morpholin-4-yl-propyl)-1H-pyrrole-3-carboxylic acid ethyl ester

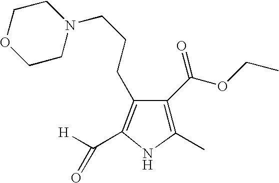

MS m/z 309 [M+1].

A-20 4-(3-Dimethylamino-propyl)-5-formyl-2-methyl-1H-pyrrole-3-carboxylic acid ethyl ester

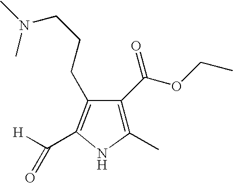

To a suspension of 4-(2-carboxy)ethyl-3-ethoxycarbonyl-2-methylpyrrole (2 g, 8.88 mmol) (Bulter, A. R., and George, S. D. (1993) Tetrahedron 49: 7017-7026) in 18 mL of dimethylformamide was added 1.73 g (10.65 mmol) of carbonyldiimidazole followed by the dropwise addition of 8.9 mL (17.76 mmol) of 2 M dimethylamine in tetrahydrofuran. After stirring for 2 hours, the reaction was diluted with water (200 mL) and cooled. The precipitate was collected by vacuum filtration, washed with water and dried to give 1.0 g of the product as a white crystalline solid. The filtrate was extracted with ethyl acetate, the organic layer was washed with brine, dried and concentrated to give 0.9 g of the product. A total of 1.9 g (85% yield) of 4-(2-dimethylcarbamoyl-ethyl)-2-methyl-1H-pyrrole-3-carboxylic acid ethyl ester was obtained. ¹H-NMR (360 MHz, dimethylsulfoxide-d6) δ 10.89 (br s, 1H, NH), 6.40 (d, 1H), 4.13 (q, 2H, OCH₂), 2.92 (s, 3H, NCH₃), 2.79 (s, 3H, NCH₃), 2.75 (m, 2H, CH₂), 2.45 (m, 2H, CH₂), 2.35 (s, 3H, CH₃), 1.23 (t, 3H, CH₃). MS m/z 252 [M+].

To a heterogeneous mixture of 4-(2-dimethylcarbamoyl-ethyl)-2-methyl-1H-pyrrole-3-carboxylic acid ethyl ester (1.01 g, 4 mmol) in tetrahydrofuran (9 mL) was added dropwise 8 mL of borane-tetrahydrofuran complex (1 M solution in tetrahydrofuran). The mixture was heated to reflux overnight. Nine mL of methanol was added slowly to the reaction and the heating was continued for another 2 hours. The cooled reaction was quenched with 1 N hydrochloric acid and extracted with ethyl acetate. The aqueous layer was basified with aqueous potassium hydroxide, extracted with ethyl acetate, and the organic layer washed with brine, dried and concentrated to give 616 mg (65% yield) of 4-(3-dimethylamino-propyl)-2-methyl-1H-pyrrole-3-carboxylic acid ethyl ester as a faint orange oil. ¹H-NMR (360 MHz, dimethylsulfoxide-d6) δ 10.84 (br s, 1H, NH), 6.36 (d, 1H), 4.12 (q, 2H, OCH₂), 2.52 (m, 2H, CH₂), 2.34 (s, 3H, CH₃), 2.17 (m, 2H, CH₂), 2.08 (s, 6H, N(CH₃)₂), 1.57 (m, 2H, CH₂), 1.23 (t, 3H, CH₃).

MS m/z 238 [M+].

4-(3-Dimethylamino-propyl)-2-methyl-1H-pyrrole-3-carboxylic acid ethyl ester (600 mg, 2.5 mmol) was formylated using phosphorus oxychloride and dimethylformamide to give 645 mg (96% yield) of 4-(3-dimethylamino-propyl)-5-formyl-2-methyl-1H-pyrrole-3-carboxylic acid ethyl ester. ¹H-NMR (360 MHz, dimethylsulfoxide-d6) δ 12.15 (br s, 1H, NH), 9.59 (s, 1H, CHO), 4.19 (q, 2H, OCH₂), 2.93 (m, 2H, CH₂), 2.41 (s, 3H, CH₃), 2.18 (m, 2H, CH₂), 2.09 (s, 6H, N(CH₃)₂), 1.63 (m, 2H, CH₂), 1.27 (t, 3H, CH₃). MS m/z 266 [M+].

A-21 3-Methyl-5-(4-methyl-piperazine-1-carbonyl)-1H-pyrrole-2-carbaldehyde

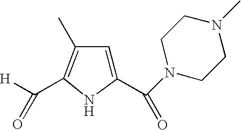

Dimethylformamide (3 mL, 6 equivalents) was cooled with stirring in an ice bath. To this was added phosphorus oxychloride (1.1 equivalents, 0.67 mL). After 30 minutes, a solution of 4-methyl-2-pyrrolecarboxylic acid ethyl ester (1 g, 6.53 mmol) in dimethylformamide (2 M, 3 mL) was added to the reaction and stirring continued. After 1 hour, the reaction was warmed to room temperature. After another 2.5 hours, the reaction mixture was diluted with water (100 mL) and basified to pH 11 with 1 N sodium hydroxide solution. The precipitate was collected by vacuum filtration, washed with water and dried to afford 0.8 g (68% yield) 5-formyl-4-methyl-1H-pyrrole-2-carboxylic acid ethyl ester as a white solid. ¹H-NMR (360 MHz, dimethylsulfoxide-d6) δ 12.6 (br s, 1H, NH), 9.78 (s, 1H, CHO), 6.68 (s, 1H, CH), 4.26 (q, 2H, OCH₂), 2.28 (s, 3H, CH₃), 1.28 (t, 3H, CH₃). MS m/Z 181 [M+].

5-Formyl-4-methyl-1H-pyrrole-2-carboxylic acid ethyl ester was dissolved in water (35 mL) and ethanol (15 mL) with stirring. Potassium hydroxide (2 equivalents, 0.5 g) was added and the mixture heated to 100° C. After 1 hour the mixture was cooled to room temperature and concentrated to about 2/3 volume. The water layer was acidified to pH 3 using 2 N hydrochloric acid. The white solid was collected by vacuum filtration and washed with water to afford 0.67 g (68% yield) of 5-formyl-4-methyl-1H-pyrrole-2-carboxylic acid as a tan solid. ¹H-NMR (360 MHz, dimethylsulfoxide-d6) δ 12.92 (br s, 1H, COOH), 12.48 (br s, 1H, NH), 9.76 (s, 1H, CHO), 6.63 (s, 1H, pyrrole CH), 2.28 (s, 3H, CH₃). MS m/z 152 [M+−1].

5-Formyl-4-methyl-1H-pyrrole-2-carboxylic acid was amidated with 1-methylpiperazine using General Amidation Procedure 1 to give 3-Methyl-5-(4-methyl-piperazine-1-carbonyl)-1H-pyrrole-2-carbaldehyde. MS m/z 236 [M+1].

A-22 5-(4-Methyl-piperazine-1-carbonyl)-1H-pyrrole-2-carbaldehyde

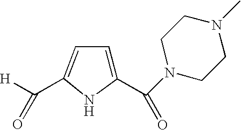

To a solution of dimethylformamide (21 mL, 0.27 mol) in 75 mL of dichloroethane was added a solution of phosphorus oxychloride (25 mL, 0.27 mol) in 75 mL of dichloroethane at 0° C. The mixture was stirred at room temperature for 30 minutes and cooled to 0° C. To the mixture was added a solution of ethyl pyrrole-2-carboxylate (25 g, 0.18 mol) in 50 mL of dichloroethane dropwise. The resulting mixture was stirred at room temperature for 30 minutes and then at 40° C. for 1 hour. The mixture was poured into ice and basified to pH 11 with 5 N sodium hydroxide solution. The mixture was extracted with ethyl acetate and the extract washed with water, brine and dried over anhydrous sodium sulfate. The two products were separated by column chromatography (1:3 ethyl acetate:hexane) to give 15 g (50% yield) of 5-formyl-1H-pyrrole-2-carboxylic acid ethyl ester and 2 g (7% yield) of 4-formyl-1H-pyrrole-2-carboxylic acid ethyl ester. ¹H-NMR (5-formyl-1H-pyrrole-2-carboxylic acid ethyl ester) (300 MHz, dimethylsulfoxide) δ 13.02 (br s, 1H, NH), 9.69 (s, 1H, CHO), 6.95 (d, 1H), 6.86 (d, 1H), 4.27 (q, 2H, CH₃), 1.28 (t, 3H, CH₃). MS m/z 167 [M+].

Refluxing 4-formyl-1H-pyrrole-2-carboxylic acid ethyl ester in 2 equivalents of potassium hydroxide in methanol:water followed by acidification to pH 3 at 0° C. gave 4-formyl-1H-pyrrole-2-carboxylic acid as a solid. MS m/z 140 [M+1].

5-Formyl-4-methyl-1H-pyrrole-2-carboxylic acid was dissolved in dimethylformamide (0.3 M) with stirring. To this was added 1-ethyl-3-(3-dimethylaminopropylcarbodiimide hydrochloride (1.2 equivalents), 1-hydroxybenzotriazole (1.2 equivalents) followed by triethylamine (2 equivalents) and 1-methylpiperazine (1 equivalent) and the reaction stirred for 12 hours (General Amidation Procedure 1). The reaction was diluted with saturated sodium bicarbonate solution, sodium hydroxide solution, brine, solid sodium chloride and extracted twice with 10% methanol in dichloromethane. The combined organic layers were washed with brine, dried over anhydrous magnesium sulfate and concentrated. The resulting oil was re-concentrated from toluene and precipitated from diethyl ether:hexanes to afford 5-(4-methyl-piperazine-1-carbonyl)-1H-pyrrole-2-carbaldehyde as a solid. MS m/z 222 [M+1].

A-23 3-(3-Pyrrolidin-1-yl-propyl)-4,5,6,7-tetrahydro-1H-indole-2-carbaldehyde

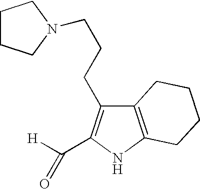

A mixture of 5-aminolevulinic acid hydrochloride (1 equivalent), 1,3-cyclohexanedione (1 equivalent) and sodium acetate (2 equivalents) in water (1 M) was heated at 110° C. for 12 hours and then cooled. The resulting solid was collected by vacuum filtration, washed with 30% ethanol in water and dried under vacuum to give 3-(4-oxo-4,5,6,7,tetrahydro-1H-indol-3-yl-propionic acid in 60% yield. ¹H-NMR (360 MHz, dimethylsulfoxide-d6) δ 11.91 (br s, 1H, COOH), 10.99 (br s, 1H, NH), 6.45 (d, 1H), 2.76 (t, 2H, CH₂), 2.69 (t, 2H, CH₂), 2.44 (t, 2H, CH₂), 2.26 (t, 2H, CH₂), 1.96 (m, 2H, CH₂). MS m/z 207 [M+].

To a suspension of 10 g of 3-(4-oxo-4,5,6,7,tetrahydro-1H-indol-3-yl-propionic acid (48 mmol) in 60 mL of dichloromethane was added 9.3 g (57.6 mmol) of carbonyldiimidazole. After stirring at room temperature for 2 hours, 12 mL (144 mmol) of pyrrolidine was added. The dark red reaction mixture was then stirred at room temperature overnight. The reaction was poured into water and the organic layer washed with brine, dried and concentrated to give 3-(3-oxo-3-pyrrolidin-1-yl-propyl)-1,5,6,7-tetrahydro-indol-4-one (12 g, 96% yield). ¹H-NMR (300 MHz, dimethylsulfoxide-d6) δ 11.05 (br s, 1H, NH), 6.46 (d, 1H), 3.35 (m, 2H, CH₂), 2.24 (m, 2H, CH₂), 2.66-2.73 (m, 4H, 2×CH₂), 2.44 (m, 2H, CH₂), 2.26 (m, 2H, CH₂), 1.96 (m, 2H, CH₂), 1.82 (m, 2H, CH₂), 1.73 (m, 2H, CH₂). MS m/Z 260 [M+].

To a suspension of 3-(3-oxo-3-pyrrolidin-1-yl-propyl)-1,5,6,7-tetrahydro-indol-4-one (5 g, 19.2 mmol) in tetrahydrofuran was added lithium aluminum hydride (2.9 g, 4 equivalents). The mixture was heated to reflux overnight and cooled to room temperature. To the reaction was added water (2.9 mL), 15% sodium hydroxide (2.9 mL) and water (2.9 ml). The mixture was stirred at room temperature for 30 minutes, filtered and the solids washed with ethyl acetate. The filtrate was concentrated to give 4.5 g (100% yield) of the product as a light yellow oil. ¹H-NMR (300 MHz, dimethylsulfoxide-d6) δ 9.82 (br s, 1H, NH), 6.22 (s, 1H), 2.2-2.5 (m, 12H, 6×CH₂), 1.5-1.64 (m, 10H, 5×CH₂). MS m/z 232 [M+].

Phosphorus oxychloride (2.0 mL, 21.2 mmol) was added dropwise to ice-cooled dimethylformamide (4.5 mL) and stirred at room temperature for 30 minutes. To the mixture at −5° C. was added a suspension of 3-(3-pyrrolidin-1-yl-propyl)-4,5,6,7-tetrahydro-1H-indole (4.5 g, 19.3 mmol) in 10 mL of dimethylformamide. The mixture was stirred at room temperature overnight. Ice cubes were added to the reaction followed by 10 N potassium hydroxide to pH 11-12. The mixture was stirred for 1 hour, extracted with ethyl acetate and the extract washed with brine and concentrated. The residue was dissolved in dichloromethane and filtered through silica gel eluting with 7% methanol in dichloromethane to give 3.8 g (76% yield) of the product. ¹H-NMR (300 MHz, diemthylsulfoxide-d6) δ 11.28 (br, s, 1H, NH), 9.38 (s, 1H, CHO), 2.59 (t, 2H, CH₂), 2.46 (m, 2H, CH₂), 2.3-2.44 (m, 8H, 4×CH₂), 1.55-1.65 (m, 10H, 5×CH₂). MS m/z 260 [M+].

A-24 3-[3-(4-Methyl-piperazin-1-yl)-propyl]-4,5,6,7-tetrahydro-1H-indole-2-carbaldehyde

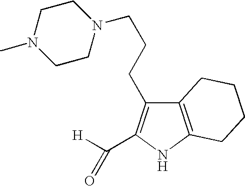

A mixture of 5-aminolevulinic acid hydrochloride (1 equivalent), 1,3-cyclohexanedione (1 equivalent) and sodium acetate (2 equivalents) in water (1 M) was heated to at 110° C. for 12 hours and then cooled. The resulting solid was collected by vacuum filtration, washed with 30% ethanol in water and dried under vacuum to give 3-(4-oxo-4,5,6,7,tetrahydro-1H-indol-3-yl-propionic acid in 60% yield. ¹H-NMR (360 MHz, DMSO-d6) δ 11.91 (br s, 1H, COOH), 10.99 (br s, 1H, NH), 6.45 (d, 1H), 2.76 (t, 2H, CH₂), 2.69 (t, 2H, CH₂), 2.44 (t, 2H, CH₂), 2.26 (t, 2H, CH₂), 1.96 (m, 2H, CH₂). MS m/z 207 [M+].

To a suspension of 10 g of 3-(4-oxo-4,5,6,7,tetrahydro-1H-indol-3-yl-propionic acid (48 mmol) in 60 mL of dichloromethane was added 9.3 g (57.6 mmol) of carbonyldiimidazole. After stirring at room temperature for 2 hours, 5.3 mL (48 mmol) of 1-methylpiperazine and 8.4 mL (48 mmol) of N,N-diisopropylethylamine were added. The dark red reaction mixture was stirred at room temperature overnight. The reaction was poured into water and the organic layer washed with brine, dried and concentrated to give 8 g (57% yield) of 3-[3-(4-methyl-piperazin-1-yl)-3-oxo-propyl]-1,5,6,7-tetrahydro-indol-4-one. ¹H-NMR (360 MHz, dimethylsulfoxide-d6) δ 10.97 (br s, 1H, NH), 6.47 (d, 1H), 3.43 (m, 4H, 2×CH₂), 2.67-2.75 (m, 4H, 2×CH₂), 2.51 (m, 2H, CH₂), 2.27 (m, 2H, CH₂), 2.20 (m, 4H, 2×CH₂), 2.15 (s, 3H, CH₃), 1.97 (m, 2H, CH₂). MS m/z 289 [M+].

To a suspension of 3-[3-(4-methyl-piperazin-1-yl)-3-oxo-propyl]-1,5,6,7-tetrahydro-indol-4-one (5 g, 17 mmol) in 300 mL of tetrahydrofuran was added dropwise a solution of lithium aluminum hydride in tetrahydrofuran (2.6 g, 68 mmol). The mixture was heated to reflux overnight. To the cooled reaction was sequentially added 2.6 mL each of water, 15% sodium hydroxide and water. The reaction was stirred at room temperature for 30 minutes and the insolubles removed by vacuum filtration. The filtrate was concentrated to give 4.5 g (100% yield) of 3-[3-(4-methyl-piperazin-1-yl)-propyl]-4,5,6,7-tetrahydro-1H-indole. ¹H-NMR (360 MHz, dimethylsulfoxide-d6) δ 9.79 (br s, 1H, NH), 6.22 (d, 1H), 2.44 (m, 2H, CH₂), 2.21-2.30 (m, 14H, 7×CH₂), 2.12 (s, 3H, CH₃), 1.65 (m, 4H, 2×CH²), 1.53 (m, 2H, CH₂). MS m/z 261 [M+].

Phosphorus oxychloride (1.8 mL, 18.9 mmol) was added dropwise to cooled dimethylformamide (3.8 mL, 51.6 mmol). After stirring at room temperature for 30 minutes, it was cooled to −5° C. and a solution of 3-[3-(4-methyl-piperazin-1-yl)-propyl]-4,5,6,7-tetrahydro-1H-indole (4.5 g, 17.2 mmol) in dimethylformamide (9 mL) was added dropwise. The mixture was stirred at room temperature overnight. The reaction was quenched with ice, followed by 10 N sodium hydroxide to pH 10-11. After stirring at room temperature for 1 hour, the reaction was extracted with ethyl acetate, washed with brine, dried and concentrated to give 3.1 g (62% yield) of 3-[3-(4-methyl-piperazin-1-yl)-propyl]-4,5,6,7-tetrahydro-1H-indole-2-carbaldehyde. ¹H-NMR (360 MHz, diemthylsulfoxide-d6) δ 11.24 (br, s, 1H, NH), 9.42 (s, 1H, CHO), 2.60 (t, 2H, CH₂), 2.51 (m, 2H, CH₂), 2.35 (m, 2H, CH₂), 2.28 (m, 8H, 4×CH₂), 2.21 (m, 2H, CH₂), 2.12 (s, 3H, CH₃), 1.57-1.68 (m, 6H, 3×CH₂). MS m/z 289 [M+].

A-25 3-(3-Morpholin-4-yl-propyl)-4,5,6,7-tetrahydro-1H-indole-2-carbaldehyde

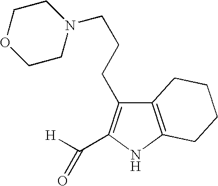

To a suspension of 1.9 g (50 mmol) of lithium aluminum hydride in 30 mL of tetrahydrofuran was added dropwise a solution of 11.3 g (43 mmol) of 1-morpholin-4-yl-3-(4,5,6,7-tetrahydro-1H-indol-3-yl)-propan-1-one in 20 mL of tetrahydrofuran. The reaction mixture was stirred at 80° C. for 2.5 hours and then cooled in an ice bath. Ice cubes were slowly added to the reaction mixture until no more gas was generated. A few drops of 2 N sodium hydroxide were added and the reaction mixture was stirred at room temperature for 30 minutes, extracted with ethyl acetate and the extract dried over anhydrous sodium sulfate and concentrated to give 9.2 g (89% yield) of 3-(3-morpholin-4-yl-propyl)-4,5,6,7-tetrahydro-1H-indole as a red oil which was used without further purification.

To an ice-cooled solution of 0.32 mL (4.1 mmol) of dimethylformamide in 3 mL of dichloromethane was added dropwise 0.4 mL (4.1 mmol) of phosphorus oxychloride. The reaction mixture was stirred at room temperature for 15 minutes and a solution of 680 mg (2.7 mmol) of 3-(3-morpholin-4-yl-propyl)-4,5,6,7-tetrahydro-1H-indole in 20 mL of dichloromethane was added dropwise at 0° C. The mixture was refluxed at 60° C. for 4 hours and cooled in an ice bath. Ice cubes were slowly added to the mixture followed by addition of 2 N sodium hydroxide to pH 12. The mixture was stirred at room temperature for 30 minutes and extracted with ethyl acetate. The organic layer was washed with brine, dried over anhydrous sodium sulfate and concentrated to give crude product which was purified on a silica gel column eluting with dichloromethane-methanol-ammonium hydroxide (98:2:1) to give 600 mg (83% yield) of 3-(3-morpholin-4-yl-propyl)-4,5,6,7-tetrahydro-1H-indole-2-carbaldehyde as a dark red oil. ¹H-NMR (360 MHz, dimethylsulfoxide-d6) δ 11.33 (s, br, 1H, NH). MS m/z 277 [M+1].

A-26 6,6-Dimethyl-3-(3-morpholin-4-yl-propyl)-4,5,6,7-tetrahydro-1H-indole-2-carbaldehyde

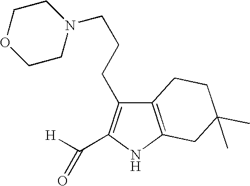

To a suspension of 1.65 g (7 mmol) of 3-(4-oxo-4,5,6,7-6,6-dimethyl-tetrahydro-1H-indol-3-yl-propionic acid) in 25 mL of dichloromethane was added 1.36 g (8.4 mmol) of carbonyldiimidazole. After stirring at room temperature for 2 hours, 0.6 mL (7 mmol) of morpholine and 1.2 mL (7 mmol) of N,N-diisopropylethylamine were added. The dark red reaction mixture was stirred at room temperature overnight. The reaction was poured into water and the organic layer washed with brine, dried and concentrated to give 1.9 g of 6,6-dimethyl-3-(3-morpholin-4-yl-3-oxo-propyl)-1,5,6,7-tetrahydro-indol-4-one as a white solid. ¹H-NMR (360 MHz, dimethylsulfoxide-d6) δ 10.94 (br s, 1H, NH), 6.49 (s, 1H), 3.42-3.50 (m, 8H, 4×CH₂), 2.74 (m, 2H, CH₂), 2.57 (s, 2H, CH₂), 2.48 (m, 2H, CH₂), 2.17 (s, 2H, CH₂), 1.00 (s, 6H, 2×CH₃). MS m/z 304 [M+].

To a suspension of 1.1 g (30 mmol) of lithium aluminum hydride in 10 mL of tetrahydrofuran was added dropwise a solution of 1.8 g (43 mmol) of 6,6-dimethyl-3-(3-morpholin-4-yl-3-oxo-propyl)-1,5,6,7-tetrahydro-indol-4-one in 10 mL of tetrahydrofuran. The mixture was stirred at 80° C. for 2.5 hours and then cooled in an ice bath. Ice cubes were added to the reaction mixture slowly until no more gas was generated. A few drops of 2 N sodium hydroxide were added and the reaction mixture was stirred at room temperature for 30 minutes, extracted with ethyl acetate, and the extract dried over anhydrous sodium sulfate and concentrated to give 1.6 g of 6,6-dimethyl-3-(3-morpholin-4-yl-propyl)-4,5,6,7-tetrahydro-1H-indole as a white solid which was used without further purification. ¹H-NMR (360 MHz, dimethylsulfoxide-d6) δ 9.75 (br s, 1H, NH), 6.24 (s, 1H), 3.54 (m, 4H, 2×CH₂), 2.23-2.31 (m, 12H, 6×CH₂), 1.58 (m, 2H, CH₂), 1.40 (m, 2H, CH₂), 0.92 (s, 6H, 2×CH₃). MS m/z 276 [M+].

6,6-Dimethyl-3-(3-morpholin-4-yl-propyl)-4,5,6,7-tetrahydro-1H-indole was formylated and recrystallized from 20% ethyl acetate in hexane to give 0.5 g of 6,6-dimethyl-3-(3-morpholin-4-yl-propyl)-4,5,6,7-tetrahydro-1H-indole-2-carbaldehyde as a light yellow solid. ¹H-NMR (360 MHz, dimethylsulfoxide-d6) δ 11.25 (br, s, 1H, NH), 9.43 (s, 1H, CHO), 3.54 (m, 4H, 2×CH₂), 2.63 (t, 2H, CH₂), 2.20-2.37 (m, 10H, 5×CH₂), 1.62 (m, 2H, CH₂), 1.45 (t, 2H, CH₂), 0.93 (s, 6H, 2×CH₃). MS m/z 304 [M+].

A-27 3,5-Dimethyl-4-(morpholine-4-carbonyl)-1H-pyrrole-2-carbaldehyde

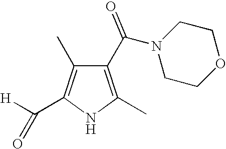

MS m/z 236 [M+1].

A-28 3,5-Dimethyl-4-[4-(4-methyl-piperazine-1-carbonyl)-phenyl]-1H-pyrrole-2-carbaldehyde

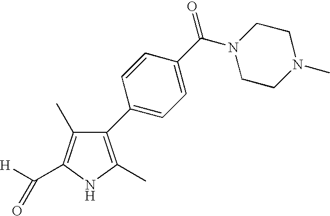

3,5-Dimethyl-1H-pyrrole-2-carboxylic acid ethyl ester (204 g), 177 g of potassium carbonate and 1428 mL of acetonitrile were stirred and cooled to 4° C. in an ice bath. N-Bromosuccinimide (228 g) was added in portions with vigorous stirring keeping the temperature below 17° C. The mixture was warmed to 20° C. and 2856 mL of water was slowly added. The mixture was stirred for 30 minutes. The precipitate was collected by vacuum filtration and washed three times with 400 mL of ethanol:water 1:2 each time. The solids were dried under vacuum at 60° C. to give 228 g (76% yield) of 4-bromo-3,5-dimethyl-1H-pyrrole-2-carboxylic acid ethyl ester as an off-white solid. ¹H-NMR (dimethylsulfoxide-d6) δ 1.26 (t, 3H, CH₃), 2.14, 2.17 (2×s, 2×3H, 2×CH₃), 4.20 (d, 2H, CH₂O), 11.75 (br s, 1H, NH). MS m/z 246,248 [M+1].

4-Bromo-3,5-dimethyl-1H-pyrrole-2-carboxylic acid ethyl ester (16.5 g), 12.5 g of 4-carboxyphenylboronic acid, 2.6 g of tetrakis(triphenylphosphine)palladium(0), 33.4 g of potassium carbonate, 169 mL of dimethylformamide and 85 mL of water were purged with nitrogen and refluxed for 18 hours at 110-112° C. A black and gray precipitate replaced the original yellow-brown solid catalyst. The mixture was cooled to room temperature and decanted, leaving black and gray solids on the walls of the flask. The mixture was vacuum filtered to remove remaining black and gray precipitate and the solids washed with 10 mL of ethanol:water 1:1. Water (1000 mL) and 15 mL of 9 N potassium hydroxide were slowly added to the combined filtrates to give a gray precipitate which was collected and discarded. The filtrate was cooled in an ice bath and acidified to pH 2.5 with 10 N hydrochloric acid and then diluted with 500 mL of water. The solids were collected by vacuum filtration, washed three times with 20 mL of water each time, and dried under vacuum at 60° C. to give 13.6 g (69% yield) of 4-(4-carboxy-phenyl)-3,5-dimethyl-1H-pyrrole-2-carboxylic acid ethyl ester as a light pink solid. ¹H-NMR (dimethylsulfoxide-d6) δ 1.28 (t, 3H, CH₃), 2.20, 2.22 (2×s, 2×3H, 2×CH₃), 4.21 (d, 2H, CH₂O), 7.34, 7.93 (multiplets, 4H, aromatic), 11.63 (br s, 1H, NH), 12.80 (br s, 1H, COOH). MS m/z 288 [M+1].

4-(4-Carboxy-phenyl)-3,5-dimethyl-1H-pyrrole-2-carboxylic acid ethyl ester (11.8 g), 11.8 mL of ethylene glycol, 36 mL of water and 13.6 mL of 9 N potassium hydroxide were refluxed at 105° C. for 25 minutes. Thin layer chromatography (ethyl acetate:hexane:acetic acid 4:6:0.5) showed conversion of all starting material at Rf 0.5 to a new product at Rf 0.3. The mixture was cooled to 98° C. and 13 mL of 10 N hydrochloric acid was added to a pH of 2.0 accompanied by rapid release of carbon dioxide gas. The maximum temperature reached was 103° C. which dropped to 95° C. by the end of the gas evolution. The mixture was cooled in an ice bath and vigorously stirred for 30 minutes. The solids were collected by vacuum filtration and washed three times with 20 mL of water each time to give 8.7 g (99% yield) of 4-(2,4-dimethyl-1H-pyrrol-3-yl)-benzoic acid as a light purple solid. ¹H-NMR (dimethylsulfoxide-d6) δ 2.00, 2.18 (2×s, 2×3H, 2×CH₃), 6.46 (s, 1H, pyrrole CH), 7.34, 7.90 (multiplets, 4H, aromatic), 10.49 (br s, 1H, NH), 12.70 (br s, 1H, COOH). MS m/z 216 [M+1].

Dimethylformamide (7.5 g) and 45 mL of dichloromethane were cooled to 6° C. and 7.3 mL of phosphorus oxychloride were added with stirring. 4-(2,4-Dimethyl-1H-pyrrol-3-yl)-benzoic acid (11.1 g) was slowly added. The mixture was refluxed for 30 minutes. Thin layer chromatography (ethyl acetate:hexane:acetic acid 4:6:0.5) showed no starting material at Rf 0.5 and a new spot at the origin. The mixture was cooled to 5° C. and 50 mL of ice water were added. Potassium hydroxide (9 N, 60 mL total) was slowly added with cooling. The final pH was 12-13. The maximum temperature reached was 48° C. Water (100 mL) was added along with 15 mL of dichloromethane, some of which had boiled off. The aqueous phase was isolated at a temperature of about 35° C. to prevent precipitation. The aqueous phase was adjusted to pH 3 by slow addition of about 21 mL of 10 N hydrochloric acid with stirring and cooling in an ice bath. The solids were collected by vacuum filtration and washed 3 times with 15 mL of water each time to give 5.6 g (50% yield) of 4-(5-formyl-2,4-dimethyl-1H-pyrrol-3-yl)-benzoic acid as a tan solid. ¹H-NMR (dimethylsulfoxide-d6) δ 2.23, 2.27 (2×s, 2×3H, 2×CH₃), 7.40, 7.95 (multiplets, 4H, aromatic), 9.56 (s, 1H, CHO), 11.85 (br s, 1H, NH), 12.90 (br s, 1H, COOH). MS m/z 244 [M+1].

4-(5-Formyl-2,4-dimethyl-1H-pyrrol-3-yl)-benzoic acid was reacted with 1-methylpiperazine using General Amidation Procedure 1 to give 3,5-dimethyl-4-[4-(4-methyl-piperazine-1-carbonyl)-phenyl]-1H-pyrrole-2-carbaldehyde. MS m/z 326 [M+1].

A-29 3,5-Dimethyl-4-[3-(morpholine-4-carbonyl)-phenyl]-1H-pyrrole-2-carbaldehyde

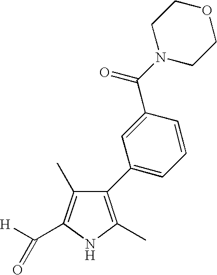

3,5-Dimethyl-1H-pyrrole-2-carboxylic acid ethyl ester (204 g), 177 g of potassium carbonate and 1428 mL of acetonitrile were stirred and cooled to 4° C. in an ice bath. N-Bromosuccinimide (228 g) was added in portions with vigorous stirring keeping the temperature under 17° C. The mixture was warmed to 20° C. and 2856 mL of water was slowly added. The precipitate was collected by vacuum filtration and washed three times with 400 mL of ethanol:water 1:2 each time. The solids were dried under vacuum at 60° C. to give 228 g (76% yield) of 4-bromo-3,5-dimethyl-1H-pyrrole-2-carboxylic acid ethyl ester as an off-white solid. ¹H-NMR (dimethylsulfoxide-d6) δ 1.26 (t, 3H, CH₃), 2.14, 2.17 (2×s, 2×3H, 2×CH₃), 4.20 (d, 2H, CH₂O), 11.75 (br s, 1H, NH). MS m/z 246, 248 [M+1].

4-Bromo-3,5-dimethyl-1H-pyrrole-2-carboxylic acid ethyl ester (23.6 g), 17.5 g of 3-carboxyphenylboronic acid, 3.6 g of tetrakis(triphenylphosphine)palladium(0), 47.1 g of potassium carbonate, 190 mL of dimethylformamide and 95 mL of water were purged with nitrogen and refluxed for 4.5 hours at 110-112° C. A black and gray precipitate replaced the original yellow-brown solid catalyst. Thin layer chromatography (ethyl acetate:hexane:acetic acid 4:6:0.5) showed a trace of starting material at Rf 0.8, the presence of product at Rf 0.6 and minor spots at Rf 0.7 and Rf 0.5. The mixture was cooled to room temperature and vacuum filtered to removed the black and gray precipitate. The filter cake was washed with 20 mL of ethanol:water 1:1. Water (1140 mL) and 20 mL of 9 N potassium hydroxide were slowly added to the combined filtrates to give a gray precipitate which was collected and washed twice with 20 mL of ethanol:water 1:1. The filtrate was cooled in an ice bath and acidified to pH 2.5 with about 85 mL of 10 N hydrochloric acid. The solids were collected by vacuum filtration, washed three times with 30 mL of water each time, and dried under vacuum at 60° C. to give 18.8 g (68% yield) of crude 4-(3-carboxy-phenyl)-3,5-dimethyl-1H-pyrrole-2-carboxylic acid ethyl ester as a light pink solid. The crude was recrystallized three times from approximately 12 mL of ethanol per gram each time to give 11.7 g (62% yield for the recrystallization) of 4-(3-carboxy-phenyl)-3,5-dimethyl-1H-pyrrole-2-carboxylic acid ethyl ester as an off-white solid. ¹H-NMR (dimethylsulfoxide-d6) δ 1.28 (t, 3H, CH₃), 2.17, 2.20 (2×s, 2×3H, 2×CH₃), 4.21 (d, 2H, CH₂O), 7.45, 7.75 (multiplets, 4H, aromatic), 11.46 (br s, 1H, NH), 12.93 (br s, 1H, COOH). MS m/z 288 [M+1].

4-(3-Carboxy-phenyl)-3,5-dimethyl-1H-pyrrole-2-carboxylic acid ethyl ester (15.0 g), 15 mL of ethylene glycol, 45 mL of water and 17.4 mL of 9 N potassium hydroxide were refluxed at 105° C. for 30 minutes. Thin layer chromatography (ethyl acetate:hexane:acetic acid 4:6:0.5) showed conversion of all starting material at Rf 0.5 to a new product at Rf 0.3. The mixture was cooled 100° C. and 21 mL of 10 N hydrochloric acid were added to a pH of 2.5 accompanied by rapid release of carbon dioxide gas. The mixture was cooled to ambient temperature and vigorously stirred for 30 minutes. The solids were collected by vacuum filtration and washed three times with 20 mL of water each time to give 10.9 g (97% yield) of 3-(2,4-dimethyl-1H-pyrrol-3-yl)-benzoic acid as a light purple solid. ¹H-NMR (dimethylsulfoxide-d6) δ 2.08, 2.16 (2×s, 2×3H, 2×CH₃), 6.45 (s, 1H, pyrrole CH), 7.46, 7.75 (multiplets, 4H, aromatic), 10.42 (br s, 1H, NH), 12.81 (br s, 1H, COOH). MS m/z 216 [M+1].

Dimethylformamide (7.5 mL) and 55 mL of dichloromethane were cooled to 3° C. and 7.2 mL of phosphorus oxychloride were added with stirring. 4-(2,4-Dimethyl-1H-pyrrol-3-yl)-benzoic acid (10.9 g) was slowly added. The mixture was refluxed for 30 minutes. Thin layer chromatography (ethyl acetate:hexane:acetic acid 4:6:0.5) showed no starting material at Rf 0.6 and a new spot at the origin. The mixture was cooled to 5° C. and 55 mL of ice water were added. Potassium hydroxide (9 N, 50 mL total) was slowly added with cooling. The final pH was 12-13. The maximum temperature reached was 48° C. Water (100 mL) was added along with 15 mL of dichloromethane to replace that which had boiled off. The aqueous phase was isolated at a temperature of about 35° C. The aqueous phase was adjusted to pH 3 by slow addition of about 21 mL of 10 N hydrochloric acid with stirring and cooling in an ice bath. The solids were collected by vacuum filtration and washed 3 times with 15 mL of water each time to give 6.1 g (50% yield) of 3-(5-formyl-2,4-dimethyl-1H-pyrrol-3-yl)-benzoic acid as a reddish solid. ¹H-NMR (dimethylsulfoxide-d6) δ 2.20, 2.24 (2×s, 2×3H, 2×CH₃), 7.53, 7.78, 7.85 (multiplets, 4H, aromatic), 9.55 (s, 1H, CHO), 11.85 (br s, 2H, NH and COOH). MS m/z 244 [M+1].

3-(5-Formyl-2,4-dimethyl-1H-pyrrol-3-yl)-benzoic acid was reacted with morpholine using General Amidation Procedure 1 to give 3,5-dimethyl-4-[3-(morpholine-4-carbonyl)-phenyl]-1H-pyrrole-carbaldehyde. MS m/z 326 [M+1].

A-30 4-(4-Hydroxy-piperidin-1-ylmethyl)-3,5-dimethyl-1H-pyrrole-2-carbaldehyde

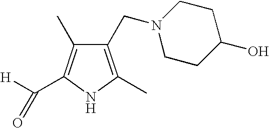

MS m/z 237 [M+1].

A-31 3,5-Diethyl-4-morpholin-4-ylmethyl-1H-pyrrole-2-carbaldehyde

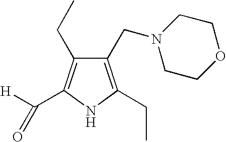

MS m/z 251 [M+1].

A-32 3,5-Dimethyl-4-[4-(morpholine-4-carbonyl)-phenyl]-1H-pyrrole-2-carbaldehyde

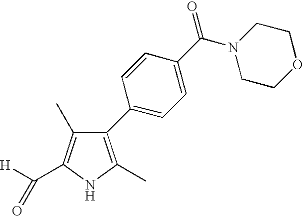

4-(5-Formyl-2,4-dimethyl-1H-pyrrol-3-yl)-benzoic acid was reacted with morpholine using General Amidation Procedure 1 to give 3,5-dimethyl-4-[4-(morpholine-4-carbonyl)-phenyl]-1H-pyrrole-2-carbaldehyde. MS m/z 313 [M+1].

A-33 3,5-Dimethyl-4-[3-(4-methyl-piperazine-1-carbonyl)-phenyl]-1H-pyrrole-2-carbaldehyde

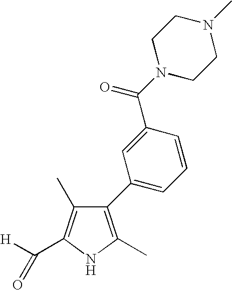

3-(5-Formyl-2,4-dimethyl-1H-pyrrol-3-yl)-benzoic acid was reacted with 1-methylpiperazine using General Amidation Procedure 1 to give 3,5-dimethyl-4-[3-(4-methyl-piperazine-1-carbonyl)-phenyl]-1H-pyrrole-2-carbaldehyde. MS m/z 326 [M+1].

A-34 3-Nitro-1H-pyrrole-2-carbaldehyde

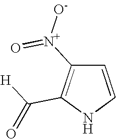

MS m/z 141 [M+1].

A-35 4-Nitro-1H-pyrrole-2-carbaldehyde

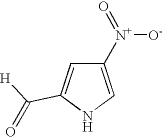

MS m/z 141 [M+1].

2. Oxindoles

Oxindoles can be prepared based on methods known in the art.

O-1 5-(6-Chloro-2,3-dihydro-indole-1-sulfonyl)-1,3-dihydro-indol-2-one

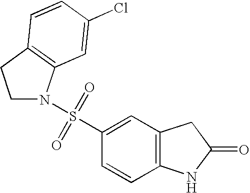

6-Chloroindole (1 g, 6.6 mmol) in glacial acetic acid (10 mL) was treated with sodium cyanoborohydride (829 mg, 13.2 mmol) portionwise at room temperature with stirring. After 1 hour, the reaction was diluted with water (25 mL) and basified with 40% sodium hydroxide with cooling. The mixture was then extracted with dichloromethane (3×50 mL), dried and concentrated to give 1 g of 6-chloroindoline. It was used in the next step without further purification. ¹HNMR (300 MHz, dimethylsulfoxide-d6) δ 6.95 (d, 1H), 6.46 (dd, 1H), 6.43 (d, 1H), 5.74 (br s, 1H, NH), 3.42 (t, 2H, CH₂), 2.85 (t, 2H, CH₂). MS m/z 349 [M+1].

O-2 5-(5-Fluoro-2,3-dihydro-indole-1-sulfonyl)-1,3-dihydro-indol-2-one

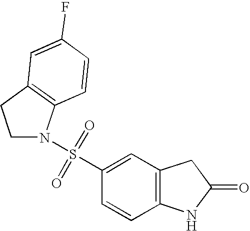

5-Fluoroindole (3 g, 22.2 mmol) in glacial acetic acid (35 mL) was treated with sodium cyanoborohydride (2.79 mg, 44.4 mmol) portionwise at room temperature with stirring. After one hour, the reaction was diluted with water and basified with 40% sodium hydroxide with cooling. The mixture was extracted 3 times with dichloromethane, dried and concentrated to give 5-fluoroindoline. It was used in the next step without further purification. ¹H-NMR (300 MHz, dimethylsulfoxide-d6) δ 6.86 (m, 1H), 6.68 (dt, 1H), 6.42 (dd, 1H), 5.32 (br s, 1H, NH), 3.38 (m, 2H, CH₂), 2.87 (t, 2H, CH₂).

A mixture of 5-fluoroindoline from above, 5-chlorosulfonyl-2-oxindole (6.1 g, 1.2 equivalent) and pyridine (7.1 mL) in dichloromethane (40 mL) was stirred at room temperature overnight. The reaction was concentrated and the residue was recrystallized from methanol to give 5 g (68% yield) of 5-(5-fluoro-2,3-dihydro-indole-1-sulfonyl)-1,3-dihydro-indol-2-one as a pink-colored solid. ¹H-NMR (360 MHz, dimethylsulfoxide-d6) δ 10.75 (br s, 1H, NH), 7.60 (m, 2H), 7.60 (dd, 1H), 7.02 (m, 2H), 6.90 (d, 1H), 3.89 (t, 2H, CH₂), 3.52 (s, 2H, CH₂), 2.87 (t, 2H, CH₂). MS 331 [M+−1].

O-3 5-(3,4-Dihydro-1H-isoquinoline-2-sulfonyl)-1,3-dihydro-indol-2-one

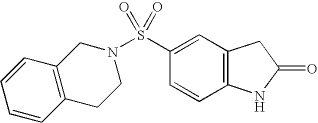

MS m/z 329 [M+1].

O-4 5-(5-Bromo-2,3-dihydro-indole-1-sulfonyl)-1,3-dihydro-indol-2-one

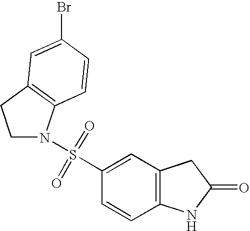

MS m/z 393 [M+1].

O-5 5-(3,4-Dihydro-2H-quinoline-1-sulfonyl)-1,3-dihydro-indol-2-one

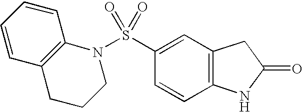

A mixture of 1,2,3,4-tetrahydroquinoline (Aldrich), 5-chlorosulfonyl-2-oxindole and pyridine in dichloromethane was stirred at room temperature overnight. The reaction was concentrated and the residue recrystallized from methanol to vie 5-(3,4,-dihydro-2H-quinoline-1-sulfonyl)-1,3-dihydro-indol-2-one. ¹H-NMR (360 MHz, dimethylsulfoxide-d6) δ 10.74 (s, 1H, NH), 7.84-7.90 (d, 1H, SO2NH), 7.67-7.72 (m, 2H, 2×CH), 7.01-7.16 (m, 4H, aromatic), 6.95-6.97(d, 1H, CH), 4.28-4.29 (m, 1H), 3.58 (s, 2H, CH₂), 2.59-2.65 (m, 2H), 1.75-1.77 (m, 2H), 1.53-1.61 (m, 2H). MS m/z 329 [M+1].

O-6 5-(2,3-Dihydro-indole-1-sulfonyl)-1,3-dihydro-indol-2-one

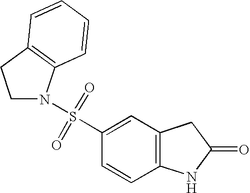

To a mixture of 5-chlorosulfonyl-2-oxindole (5 g, 21.6 mmol) and indoline (2.9 mL, 26 mmol) in tetrahydrofuran (20 mL) was added pyridine (3.4 g, 43 mmol). After stirring at room temperature for 1 day the precipitate was collected by vacuum filtration, washed with water in ethanol (20%), dried, washed with 60 mL of hot ethanol and dried under vacuum to give 7.5 g of 5-(2,3-dihydro-indole-1-sulfonyl)-1,3-dihydro-indol-2-one as a pink solid. ¹H-NMR (360 MHz, dimethylsulfoxide-d6) δ 10.76 (br s, 1H, NH), 7.62 (m, 2H), 7.43 (d, 1H), 7.13-7.18 (m, 2H), 6.95 (dt, 1H), 6.90 (d, 1H), 3.87 (t, 2H, CH₂), 3.51 (s, 2H, CH₂), 2.91 (t, 2H, CH₂). MS m/z 315 [M+1].

O-7 5-(5-Methoxy-2,3-dihydro-indole-1-sulfonyl)-1,3-dihydro-indol-2-one

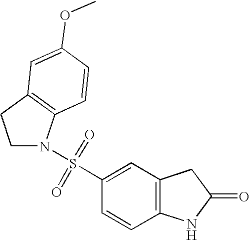

MS m/z 345 [M+1].

O-8 5-(4-Chloro-2,3-dihydro-indole-1-sulfonyl)-1,3-dihydro-indol-2-one

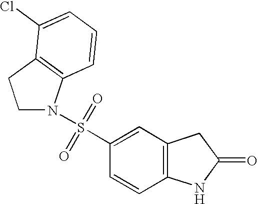

MS m/z 337 [M+1].

O-9 2-Oxo-2,3-dihydro-1H-indole-5-sulfonic acid (4-chloro-2-fluoro-phenyl)-amide

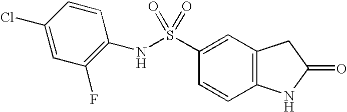

To a mixture of 5-chlorosulfonyl-2-oxindoel (5 g, 21.6 mmol) and 4-chloro-2-fluoroaniline (2.9 mL, 26 mmol) in tetrahydrofuran (30 mL) was added pyridine (3.4 g, 43 mmol). The mixture was stirred at room temperature for 1 day. The precipitate was collected by vacuum filtration, washed with water in methanol (20%), dried, washed with 60 mL of hot ethanol and dried to give 6.3 g (85%) of 2-oxo-2,3-dihydro-1H-indole-5-sulfonic acid (4-chloro-2-fluoro-phenyl)-amide as a light pink-colored solid. ¹H-NMR (300 MHz, dimethylsulfoxide-d6) δ 10.79 (br s, 1H, NH), 10.12 (br s, 1H, NH), 7.54 (m, 2H), 7.39 (dd, 1H), 7.17-7.27 (m, 2H), 6.90 (d, 1H), 3.54 (s, 2H, CH₂). MS m/z 340 [M+].

O-10 2-Oxo-2,3-dihydro-1H-indole-5-sulfonic acid amide

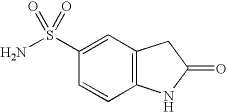

To a 100 mL flask charged with 27 mL of chlorosulfonic acid was added slowly 13.3 g of 2-oxindole. The reaction temperature was maintained below 30° C. during the addition. After the addition the reaction mixture was stirred at room temperature for 1.5 hour, heated to 68° C. for 1 hour, cooled, and poured into water. The precipitate was washed with water and dried in a vacuum oven to give 11.0 g (50% yield) of 5-chlorosulfonyl-2-oxindole which was used without further purification.

5-Chlorosulfonyl-2-oxindole (2.1 g) was added to 10 mL of ammonium hydroxide in 10 mL of ethanol and stirred at room temperature overnight. The mixture was concentrated and the solid collected by vacuum filtration to give 0.4 g (20% yield) of 5-aminosulfonyl-2-oxindole as an off-white solid. ¹H-NMR (360 MHz, dimethylsulfoxide-d6) δ10.67 (s, 1H, NH), 7.63-7.66 (m, 2H), 7.13 (s, 2H, 5-SO₂NH₂), 6.91 (d, 1H), 3.56 (s, 2H, CH₂). MS m/z 211 [M-1]+.

O-11 2-Oxo-2,3-dihydro-1H-indole-5-sulfonic acid methylamide

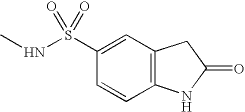

A suspension of 3.38 g of 5-chlorosulfonyl-2-oxindole in 10 mL of 2 M methylamine in tetrahydrofuran was stirred at room temperature for 4 hours. The precipitate was collected by vacuum filtration, washed twice with 5 mL of water each time and dried under vacuum at 40° C. overnight to give 3.0 g (88% yield) of 5-methylaminosulfonyl-2-oxindole. ¹H-NMR (300 MHz, DMSO-d6) δ 10.87 (br s, 1H, NH), 7.86 (br s, 1H, SO₂NH), 7.61 (d, 1H), 7.32 (d, 1H), 6.97 (d, 1H), 2.53 (s, 2H, CH₂), 2.36 (s, 3H, NHCH₃). MS m/z 226 [M].

O-12 2-Oxo-2,3-dihydro-1H-indole-5-sulfonic acid dimethylamide

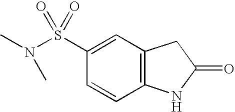

A suspension of 2.3 g of 5-chlorosulfonyl-2-oxindole in 10 mL of 2 M dimethylamine in methanol was stirred at room temperature for 4 hours. The precipitate was collected by vacuum filtration, washed with 5 mL of 1 N sodium hydroxide and 5 mL of 1 N hydrochloric acid and dried under vacuum at 40° C. overnight to give 1.9 g (79% yield) of 5-dimethylaminosulfonyl-2-oxindole. ¹H-NMR (300 MHz, DMSO-d6) δ 10.87 (br s, 1H, NH), 7.73 (d, 1H, CH), 7.58 (dd, 1H, CH), 7.02 (d, 1H, CH), 2.59 (s, 3H, CH₃), 2.54 (s, 2H, CH₂), 2.36 (s, 3H, CH₃). MS m/z 241 [M+1].

O-13 2-Oxo-2,3-dihydro-1H-indole-5-sulfonic acid thiazol-2-ylamide

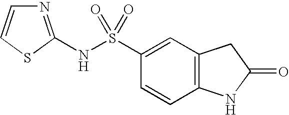

MS m/z 295 [M+1].

### Synthesis of Amidosulfonyl-Indolinones Indolinones

General Condensation Method for Condensed Indolinones

1. Condensation of an Oxindole and an Aldehyde Containing an Acid Group

A mixture of an appropriately substituted oxindole, an appropriately substituted aldehyde (1 equivalent) and piperidine (excess) in ethanol (0.2 M) was stirred at between room temperature and 100° C. After completion, the mixture was concentrated and then triturated with dilute hydrochloric acid solution. The resulting precipitate was collected by vacuum filtration, washed with water and dried to give the desired product.

2. Condensation of an Oxindole and an Aldehyde not Containing an Acid Group

A mixture of an appropriately substituted oxindole, an appropriately substituted aldehyde (1 equivalent) and piperidine (catalytic amount) in ethanol (0.2 M) was stirred at between room temperature and 100° C. After completion, the reaction was cooled to room temperature and the resulting precipitate was collected by vacuum filtration, washed with ethanol and dried to give the desired product.

The following examples were prepared using Condensation Method 1 or 2.

### Example 1

### 5-(6-Chloro-2,3-dihydro-indole-1-sulfonyl)-3-[1-[3,5-dimethyl-4-(piperazine-1-carbonyl)-1H-pyrrol-2-yl]-meth-(Z)-ylidene]-1,3-dihydro-indol-2-one FW 566.08

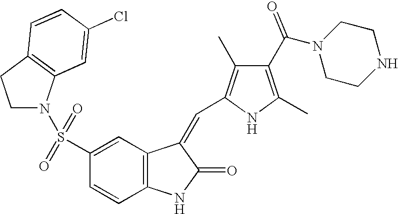

MS m/z 566[M+1].

### Example 2

### 5-(6-Chloro-2,3-dihydro-indole-1-sulfonyl)-3-[1-[4-(3,5-dimethyl-piperazine-1-carbonyl)-3,5-dimethyl-1H-pyrrol-2-yl]-meth-(Z)-ylidene]-1,3-dihydro-indol-2-one FW 594.14

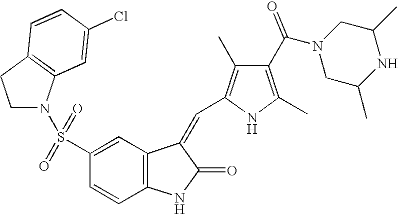

MS m/z 594 [M+1].

### Example 3

### 3-[1-[4-(3,5-Dimethyl-piperazine-1-carbonyl)-3,5-dimethyl-1H-pyrrol-2-yl]-meth-(Z)-ylidene]-5-(5-fluoro-2,3-dihydro-indole-1-sulfonyl)-1,3-dihydro-indol-2-one FW 577.68

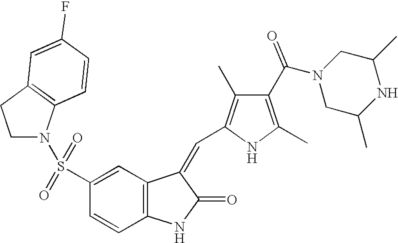

MS m/z 578 [M+1].

### Example 4

### 5-(5-Fluoro-2,3-dihydro-indole-1-sulfonyl)-3-[1-{4-[4-(2-hydroxy-ethyl)-piperazine-1-carbonyl]-3,5-dimethyl-1H-pyrrol-2-yl}-meth-(Z)-ylidene]-1,3-dihydro-indol-2-one FW 593.68

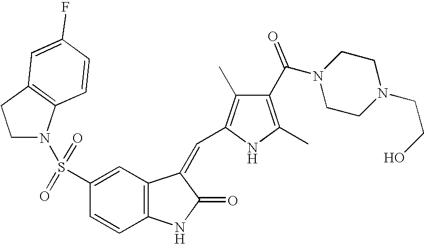

MS m/z 594 [M+1].

### Example 5

### 3-[1-[3,5-Dimethyl-4-(piperazine-1-carbonyl)-1H-pyrrol-2-yl]-meth-(Z)-ylidene]-5-(5-fluoro-2,3-dihydro-indole-1-sulfonyl)-1,3-dihydro-indol-2-one FW 549.63

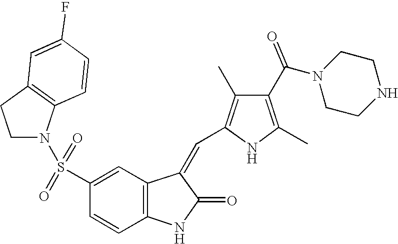

MS m/z 550 [M+1].

### Example 6

### 5-[5-(3,4-Dihydro-2H-quinoline-1-sulfonyl)-2-oxo-1,2-dihydro-indol-(3Z)-ylidenemethyl]-2,4-dimethyl-1H-pyrrole-3-carboxylic acid [3-(4-methyl-piperazin-1-yl)-propyl]-amide FW 616.79

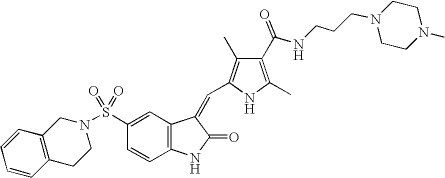

MS m/z 617 [M+1].

### Example 7

### 5-[5-(5-Bromo-2,3-dihydro-indole-1-sulfonyl)-2-oxo-1,2-dihydro-indol-(3Z)-ylidenemethyl]-2,4-dimethyl-1H-pyrrole-3-carboxylic acid [3-(4-methyl-piperazin-1-yl)-propyl]-amide FW 681.66

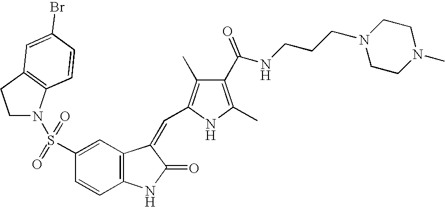

MS m/z 681 [M+1].

### Example 8

### 5-[5-(3,4-Dihydro-2H-quinoline-1-sulfonyl)-2-oxo-1,2-dihydro-indol-(3Z)-ylidenemethyl]-2,4-dimethyl-1H-pyrrole-3-carboxylic acid [3-(4-methyl-piperazin-1-yl)-propyl]-amide FW 616.79

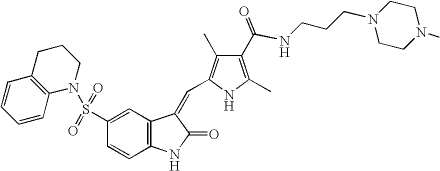

MS m/z 617 [M+1].

### Example 9

### 5-[5-(2,3-Dihydro-indole-1-sulfonyl)-2-oxo-1,2-dihydro-indol-(3Z)-ylidenemethyl]-1H-pyrrole-2-carboxylic acid FW 435.46

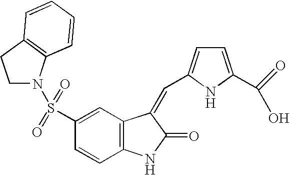

MS m/z 436 [M+1].

### Example 10

### 5-[5-(3,4-Dihydro-2H-quinoline-1-sulfonyl)-2-oxo-1,2-dihydro-indol-(3Z)-ylidenemethyl]-1H-pyrrole-2-carboxylic acid FW 449.49

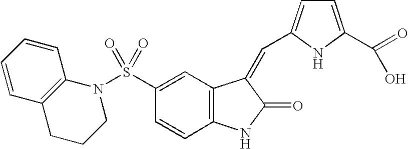

MS m/z 450 [M+1].

### Example 11

### 5-[5-(3,4-Dihydro-1H-isoquinoline-2-sulfonyl)-2-oxo-1,2-dihydro-indol-(3Z)-ylidenemethyl]-1H-pyrrole-2-carboxylic acid FW 449.49

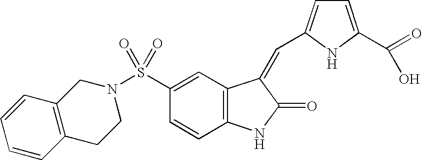

MS m/z 450 [M+1].

### Example 12

### 5-[5-(5-Bromo-2,3-dihydro-indole-1-sulfonyl)-2-oxo-1,2-dihydro-indol-(3Z)-ylidenemethyl]-1H-pyrrole-2-carboxylic acid FW 514.36

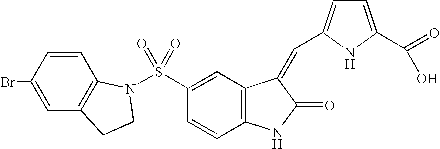

MS m/z 514 [M+1].

### Example 13

### 3-[1-[3,5-Dimethyl-4-(4-methyl-piperazine-1-carbonyl)-1H-pyrrol-2-yl]-meth-(Z)-ylidene]-5-(5-methoxy-2,3-dihydro-indole-1-sulfonyl)-1,3-dihydro-indo-1-2-one FW 575.69

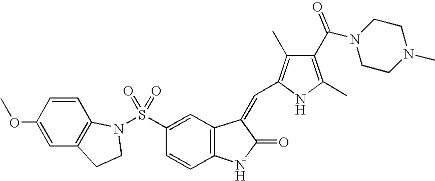

MS m/z 576 [M+1].

### Example 14

### 3-[1-[3,5-Dimethyl-4-(3-morpholin-4-yl-propyl)-1H-pyrrol-2-yl]-meth-(Z)-ylidene]-5-(5-methoxy-2,3-dihydro-indole-1-sulfonyl)-1,3-dihydro-indol-2-one FW 576.72

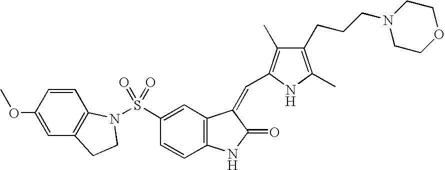

MS m/z 577 [M+1].

### Example 15

### 5-(4-Chloro-2,3-dihydro-indole-1-sulfonyl)-3-[1-[3-(2-hydroxy-ethyl)-4-(4-methyl-piperazine-1-carbonyl)-1H-pyrrol-2-yl]-meth-(Z)-ylidene]-1,3-dihydro-indol-2-one FW 596.11

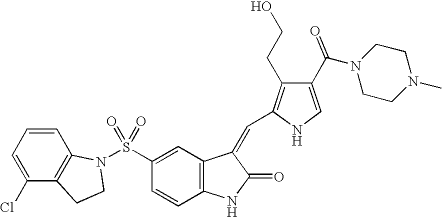

MS m/z 596 [M+1].

### Example 16

### 2-{5-[5-(2,3-Dihydro-indole-1-sulfonyl)-2-oxo-1,2-dihydro-indol-(3Z)-ylidenemethyl]-2,4-dimethyl-1H-pyrrol-3-yl}-N-(2-pyrrolidin-1-yl-ethyl)-acetamide FW 573.72

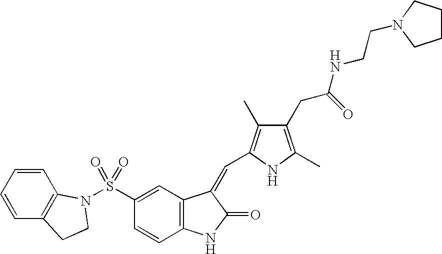

MS m/z 574 [M+1].

### Example 17

### 5-[5-(5-Methoxy-2,3-dihydro-indole-1-sulfonyl)-2-oxo-1,2-dihydro-indol-(3Z)-ylidenemethyl]-2,4-dimethyl-1H-pyrrole-3-carboxylic acid (2-diethylamino-ethyl)-amide FW 591.74

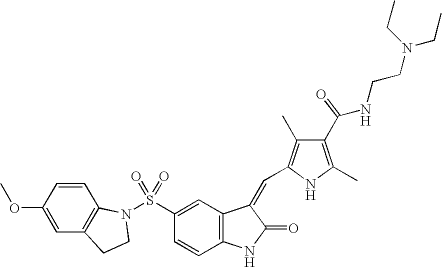

MS m/z 592 [M+1].

### Example 18

### N-(2-Diethylamino-ethyl)-2-{5-[5-(2,3-dihydro-indole-1-sulfonyl)-2-oxo-1,2-dihydro-indol-(3Z)-ylidenemethyl]-2,4-dimethyl-1H-pyrrol-3-yl}-acetamide FW 575.74

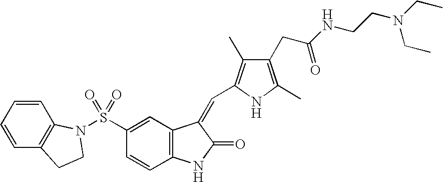

MS m/z 576 [M+1].

### Example 19

### 5-(2,3-Dihydro-indole-1-sulfonyl)-3-[1-[3-(3-dimethylamino-propyl)-6,6-dimethyl-4,5,6,7-tetrahydro-1H-indol-2-yl]-meth-(Z)-ylidene]-1,3-dihydro-indol-2-one FW 558.75

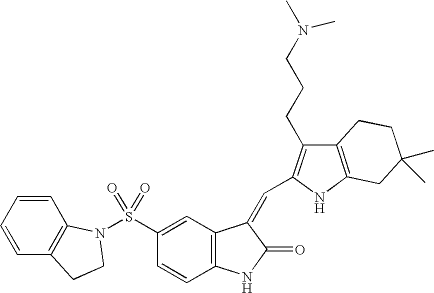

MS m/z 559 [M+1].

### Example 20

### 5-(2,3-Dihydro-indole-1-sulfonyl)-3-[1-[3,5-dimethyl-4-(piperazine-1-carbonyl)-1H-pyrrol-2-yl]-meth-(Z)-ylidene]-1,3-dihydro-indol-2-one FW 531.64

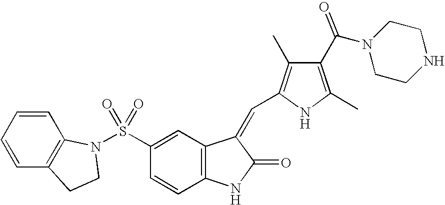

MS m/z 532 [M+1].

### Example 21

### 2-{5-[5-[(3-Chloro-phenyl)-methyl-sulfamoyl]-2-oxo-1,2-dihydro-indol-(3Z)-ylidenemethyl]-2,4-dimethyl-1H-pyrrol-3-yl}-N-(2-morpholin-4-yl-ethyl)-acetamide FW 612.15

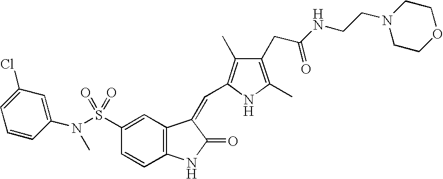

MS m/z 612 [M+1].

### Example 22

### 3-[1-[3-(2-Hydroxy-ethyl)-4-(4-methyl-piperazine-1-carbonyl)-1H-pyrrol-2-yl]-meth-(Z)-ylidene]-5-(5-methoxy-2,3-dihydro-indole-1-sulfonyl)-1,3-dihydro-indol-2-one FW 591.69

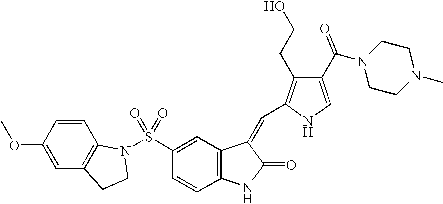

MS m/z 592 [M+1].

### Example 23

### 2-{5-[5-(2,3-Dihydro-indole-1-sulfonyl)-2-oxo-1,2-dihydro-indol-(3Z)-ylidenemethyl]-2,4-dimethyl-1H-pyrrol-3-yl}-N-(2-morpholin-4-yl-ethyl)-acetamide FW 589.72

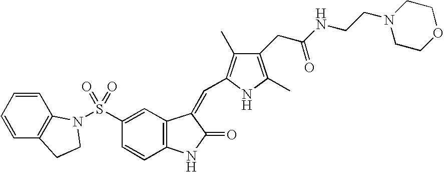

MS m/z 590 [M+1].

### Example 24

### 3-[1-[3-(3-Diethylamino-propyl)-6,6-dimethyl-4,5,6,7-tetrahydro-1H-indol-2-yl]-meth-(Z)-ylidene]-5-(2,3-dihydro-indole-1-sulfonyl)-1,3-dihydro-indol-2-one FW 586.80

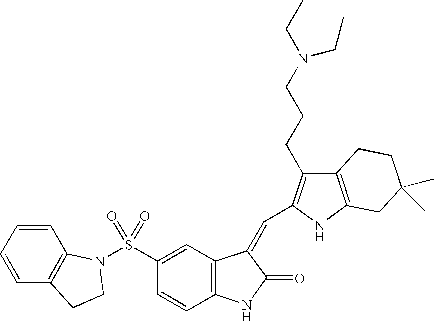

MS m/z 587 [M+1].

### Example 25

### 5-[5-(2,3-Dihydro-indole-1-sulfonyl)-2-oxo-1,2-dihydro-indol-(3Z)-ylidenemethyl]-2,4-dimethyl-1H-pyrrole-3-carboxylic acid (2-diethylamino-ethyl)-amide FW 561.71

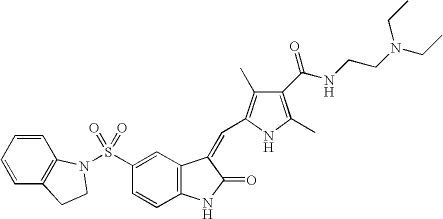

MS m/z 562 [M+1].

### Example 26

### 5-[5-(2,3-Dihydro-indole-1-sulfonyl)-2-oxo-1,2-dihydro-indol-(3Z)-ylidenemethyl]-2,4-dimethyl-1H-pyrrole-3-carboxylic acid (2-pyrrolidin-1-yl-ethyl)-amide FW 559.69

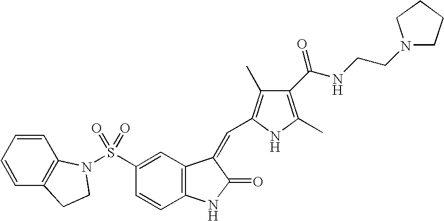

MS m/z 560 [M+1].

### Example 27

### 5-(4-Chloro-2,3-dihydro-indole-1-sulfonyl)-3-[1-[3,5-dimethyl-4-(4-methyl-piperazine-1-carbonyl)-1H-pyrrol-2-yl]-meth-(Z)-ylidene]-1,3-dihydro-indol-2-one FW 580.11

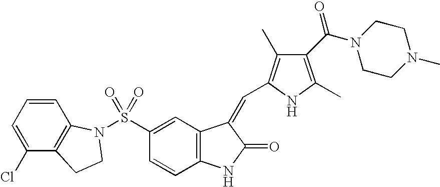

MS m/z 580 [M+1].

### Example 28

### 5-(2,3-Dihydro-indole-1-sulfonyl)-3-[1-[3,5-dimethyl-4-(4-methyl-piperazine-1-carbonyl)-1H-pyrrol-2-yl]-meth-(Z)-ylidene]-1,3-dihydro-indol-2-one FW 545.67

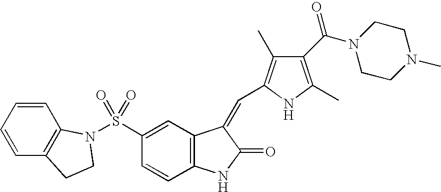

MS m/z 546 [M+1].

### Example 29

### 5-[5-(2,3-Dihydro-indole-1-sulfonyl)-2-oxo-1,2-dihydro-indol-(3Z)-ylidenemethyl]-2-ethyl-4-methyl-1H-pyrrole-3-carboxylic acid (2-pyrrolidin-1-yl-ethyl)-amide FW 573.72

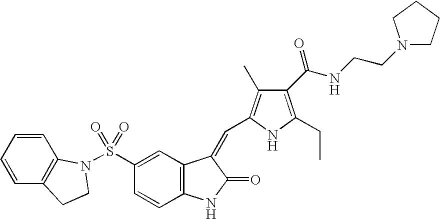

MS m/z 574 [M+1].

### Example 30

### 5-[5-(2,3-Dihydro-indole-1-sulfonyl)-2-oxo-1,2-dihydro-indol-(3Z)-ylidenemethyl]-2,4-dimethyl-1H-pyrrole-3-carboxylic acid [3-(4-methyl-piperazin-1-yl)-propyl]-amide FW 602.76

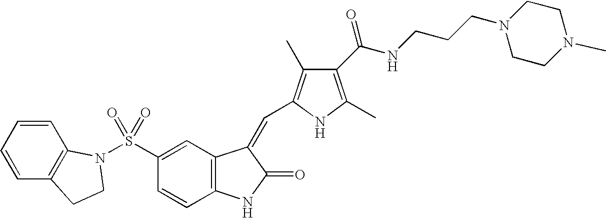

MS m/z 603 [M+l].

### Example 31

### 5-[5-(2,3-Dihydro-indole-1-sulfonyl)-2-oxo-1,2-dihydro-indol-(3Z)-ylidenemethyl]-2-methyl-4-[3-(4-methyl-piperazin-1-yl)-propyl]-1H-pyrrole-3-carboxylic acid ethyl ester FW 617.77

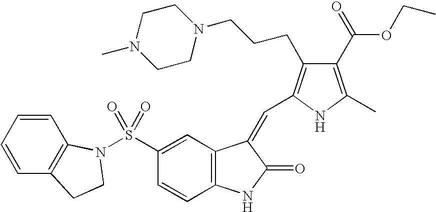

MS m/z 618 [M+1].

### Example 32

### 5-[5-(2,3-Dihydro-indole-1-sulfonyl)-2-oxo-1,2-dihydro-indol-(3Z)-ylidenemethyl]-2-methyl-4-(3-morpholin-4-yl-propyl)-1H-pyrrole-3-carboxylic acid ethyl ester FW 604.73

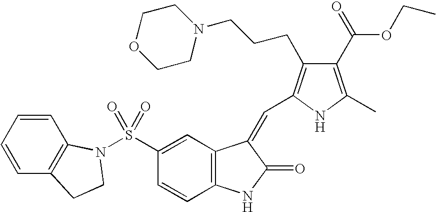

MS m/z 605 [M+1].

### Example 33

### 5-[5-(2,3-Dihydro-indole-1-sulfonyl)-2-oxo-1,2-dihydro-indol-(3Z)-ylidenemethyl]4-(3-dimethylamino-propyl)-2-methyl-1H-pyrrole-3-carboxylic acid ethyl ester FW 562.69

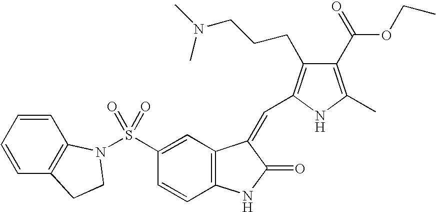

MS m/z 563 [M+1].

### Example 34

### 5-(2,3-Dihydro-indole-1-sulfonyl)-3-[1-[3-methyl-5-(4-methyl-piperazine-1-carbonyl)-1H-pyrrol-2-yl]-meth-(Z)-ylidene]-1,3-dihydro-indol-2-one FW 531.64

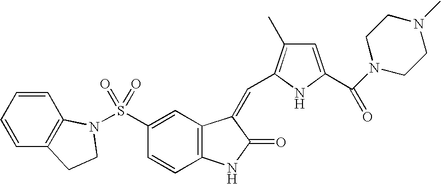

MS m/z 532 [M+1].

### Example 35

### 5-(2,3-Dihydro-indole-1-sulfonyl)-3-[1-[5-(4-methyl-piperazine-1-carbonyl)-1H-pyrrol-2-yl]-meth-(Z)-ylidene]-1,3-dihydro-indol-2-one FW 517.61

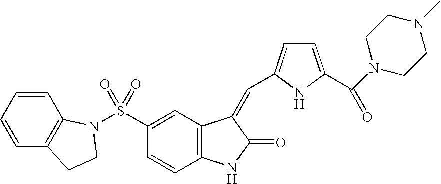

MS m/z 518 [M+1].

### Example 36

### 5-(2,3-Dihydro-indole-1-sulfonyl)-3-[1-[3-(3-pyrrolidin-1-yl-propyl)-4,5,6,7-tetrahydro-1H-indol-2-yl]-meth-(Z)-ylidene]-1,3-dihydro-indol-2-one FW 556.73

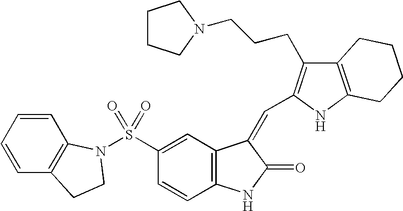

MS m/z 557 [M+1].

### Example 37

### 5-(2,3-Dihydro-indole-1-sulfonyl)-3-[1-[3-[3-(4-methyl-piperazin-1-yl)-propyl]-4,5,6,7-tetrahydro-1H-indol-2-yl]-meth-(Z)-ylidene]-1,3-dihydro-indol-2-one FW 585.77

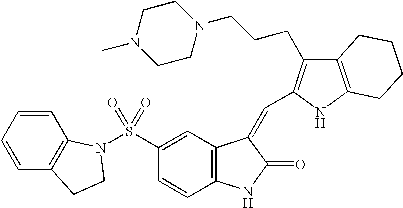

MS m/z 586 [M+1].

### Example 38

### 5-(2,3-Dihydro-indole-1-sulfonyl)-3-[1-[3-(3-morpholin-4-yl-propyl)-4,5,6,7-tetrahydro-1H-indol-2-yl]-meth-(Z)-ylidene]-1,3-dihydro-indol-2-one FW 572.73

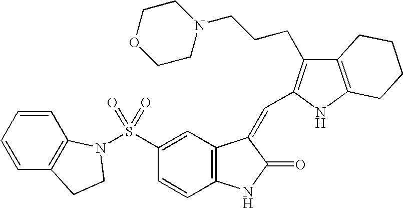

MS m/z 573 [M+1].

### Example 39

### 5-(2,3-Dihydro-indole-1-sulfonyl)-3-[1-[6,6-dimethyl-3-(3-morpholin-4-yl-propyl)4,5,6,7-tetrahydro-1H-indol-2-yl]-meth-(Z)-ylidene]-1,3-dihydro-indol-2-one FW 600.79

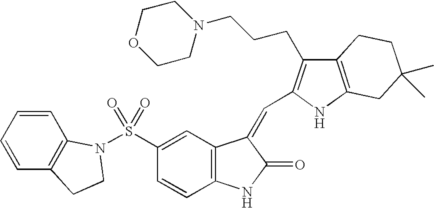

MS m/z 601 [M+1].

### Example 40

### 5-(3,4-Dihydro-1H-isoquinoline-2-sulfonyl)-3-[1-[3,5-dimethyl-4-(4-methyl-piperazine-1-carbonyl)-1H-pyrrol-2-yl]-meth-(Z)-ylidene]-1,3-dihydro-indol-2-one FW 559.69

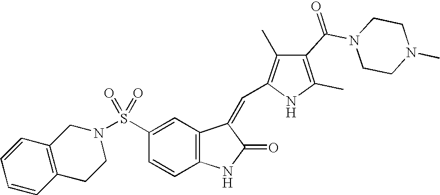

MS m/z 560 [M+1].

### Example 41

### 5-(5-Bromo-2,3-dihydro-indole-1-sulfonyl)-3-[1-[3,5-dimethyl-4-(4-methyl-piperazine-1-carbonyl)-1H-pyrrol-2-yl]-meth-(Z)-ylidene]-1,3-dihydro-indol-2-one FW 624.56

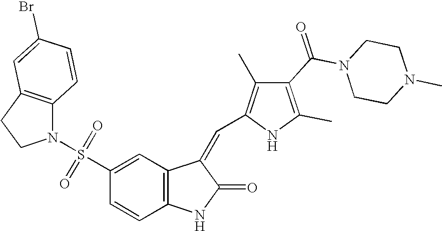

MS m/z 624 [M+1].

### Example 42

### 5-(3,4-Dihydro-2H-quinoline-1-sulfonyl)-3-[1-[3,5-dimethyl-4-(4-methyl-piperazine-1-carbonyl)-1H-pyrrol-2-yl]-meth-(Z)-ylidene]-1,3-dihydro-indol-2-one FW 559.69

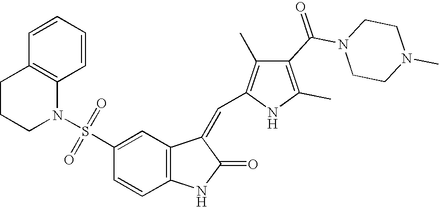

MS m/z 560 [M+1].

### Example 43

### 5-[5-(5-Bromo-2,3-dihydro-indole-1-sulfonyl)-2-oxo-1,2-dihydro-indol-(3Z)-ylidenemethyl]-2-methyl-4-[3-(4-methyl-piperazin-1-yl)-propyl]-1H-pyrrole-3-carboxylic acid ethyl ester FW 696.67

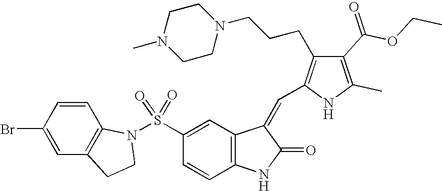

MS m/z 696 [M+1].

### Example 44

### 5-[5-(3,4-Dihydro-1H-isoquinoline-2-sulfonyl)-2-oxo-1,2-dihydro-indol-(3Z)-ylidenemethyl]-2-methyl-4-[3-(4-methyl-piperazin-1-yl)-propyl]-1H-pyrrole-3-carboxylic acid ethyl ester FW 631.80

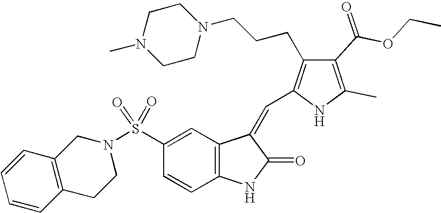

MS m/z 632 [M+1].

### Example 45

### 5-[5-(3,4-Dihydro-2H-quinoline-1-sulfonyl)-2-oxo-1,2-dihydro-indol-(3Z)-ylidenemethyl]-2-methyl-4-[3-(4-methyl-piperazin-1-yl)-propyl]-1H-pyrrole-3-carboxylic acid ethyl ester FW 631.80

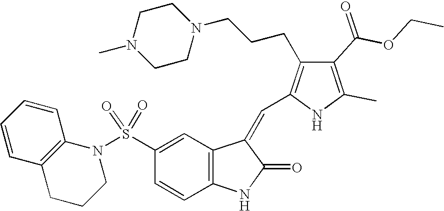

MS m/z 632 [M+1].

### Example 46

### 5-(6-Chloro-2,3-dihydro-indole-1-sulfonyl)-3-[1-[3,5-dimethyl-4-(4-methyl-piperazine-1-carbonyl)-1H-pyrrol-2-yl]-meth-(Z)-ylidene]-1,3-dihydro-indol-2-one FW 580.11

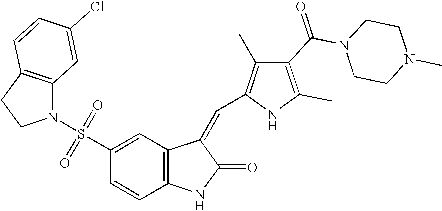

MS m/z 580 [M+1].

### Example 47

### 5-(6-Chloro-2,3-dihydro-indole-1-sulfonyl)-3-[1-[3,5-dimethyl-4-(morpholine-4-carbonyl)-1H-pyrrol-2-yl]-meth-(Z)-ylidene]-1,3-dihydro-indol-2-one FW 567.07

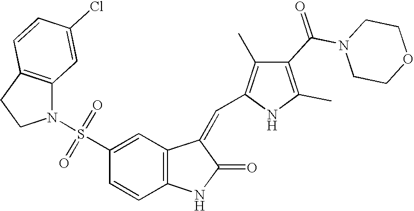

MS m/z 567 [M+1].

### Example 48

### 3-[1-[3,5-Dimethyl-4-(4-methyl-piperazine-1-carbonyl)-1H-pyrrol-2-yl]-meth-(Z)-ylidene]-5-(5-fluoro-2,3-dihydro-indole-1-sulfonyl)-1,3-dihydro-indol-2-one FW 563.66

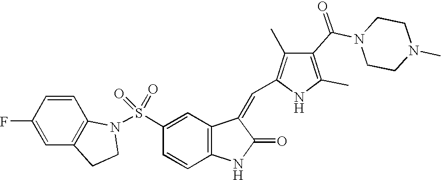

MS m/z 564 [M+1].

### Example 49

### 3-[1-[3,5-Dimethyl-4-(morpholine-4-carbonyl)-1H-pyrrol-2-yl]-meth-(Z)-ylidene]-5-(5-fluoro-2,3-dihydro-indole-1-sulfonyl)-1,3-dihydro-indol-2-one FW 550.61

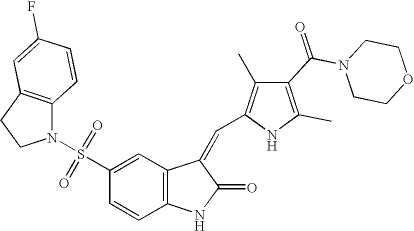

MS m/z 551 [M+1].

### Example 50

### 5-[5-(5-Fluoro-2,3-dihydro-indole-1-sulfonyl)-2-oxo-1,2-dihydro-indol-(3Z)-ylidenemethyl]-2,4-dimethyl-1H-pyrrole-3-carboxylic acid (2-diethylamino-ethyl)-amide FW 579.70

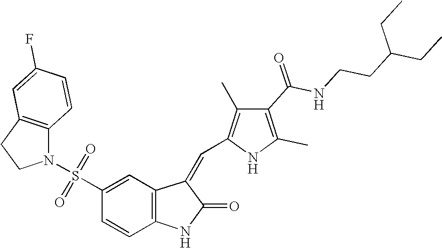

MS m/z 580 [M+1].

### Example 51

### 5-(5-Bromo-2,3-dihydro-indole-1-sulfonyl)-3-[1-[3-(3-morpholin-4-yl-propyl)-4,5,6,7-tetrahydro-1H-indol-2-yl]-meth-(Z)-ylidene]-1,3-dihydro-indol-2-one FW 651.63

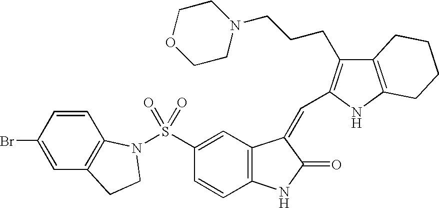

MS m/z 651 [M+1].

### Example 52

### 5-(3,4-Dihydro-1H-isoquinoline-2-sulfonyl)-3-[1-[3-(3-morpholin-4-yl-propyl)4,5,6,7-tetrahydro-1H-indol-2-yl]-meth-(Z)-ylidene]-1,3-dihydro-indol-2-one FW 586.76

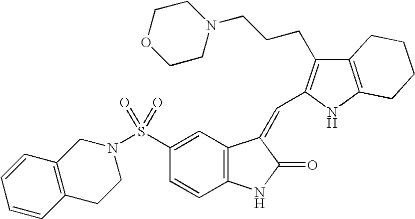

MS m/z 587 [M+1].

### Example 53

### 5-(3,4-Dihydro-2H-quinoline-1-sulfonyl)-3-[1-[3-(3-morpholin-4-yl-propyl)-4,5,6,7-tetrahydro-1H-indol-2-yl]-meth-(Z)-ylidene]-1,3-dihydro-indol-2-one FW 586.76

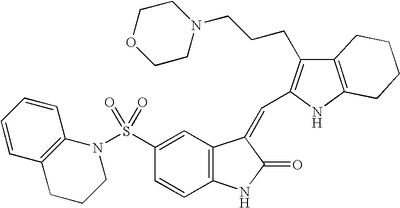

MS m/z 587 [M+1].

### Example 54

### 5-(6-Chloro-2,3-dihydro-indole-1-sulfonyl)-3-[1-{4-[4-(2-hydroxy-ethyl)-piperazine-1-carbonyl]-3,5-dimethyl-1H-pyrrol-2-yl}-meth-(Z)-ylidene]-1,3-dihydro-indol-2-one FW 610.14

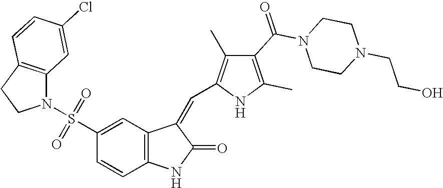

MS m/z 610 [M+1].

### Example 55

### 2-{2,4-Dimethyl-5-[2-oxo-5-sulfamoyl-1,2-dihydro-indol-(3Z)-ylidenemethyl]-1H-pyrrol-3-yl}-N-(2-pyrrolidin-1-yl-ethyl)-acetamide FW 471.58

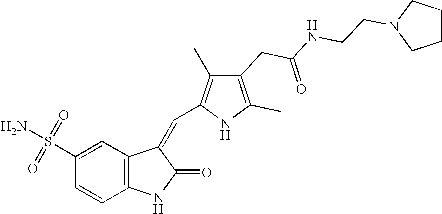

MS m/z 472 [M+1].

### Example 56

### 2-{2,4-Dimethyl-5-[5-methylsulfamoyl-2-oxo-1,2-dihydro-indol-(3Z)-ylidenemethyl]-1H-pyrrol-3-yl}-N-(2-pyrrolidin-1-yl-ethyl)-acetamide FW 485.61

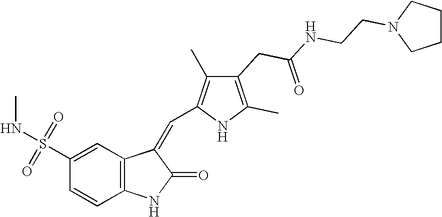

MS m/z 486 [M+1].

### Example 57

### 2-{5-[5-Dimethylsulfamoyl-2-oxo-1,2-dihydro-indol-(3Z)-ylidenemethyl]-2,4-dimethyl-1H-pyrrol-3-yl}-N-(2-pyrrolidin-1-yl-ethyl)-acetamide FW 499.64

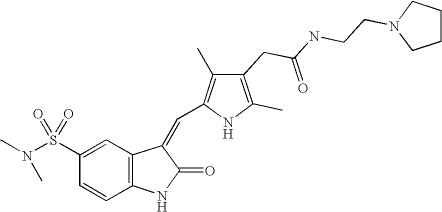

MS m/z 500 [M+1].

### Example 58

### 2-{5-[5-(2,3-Dihydro-indole-1-sulfonyl)-2-oxo-1,2-dihydro-indol-(3Z)-ylidenemethyl]-2,4-dimethyl-1H-pyrrol-3-yl}-N-(2-pyrrolidin-1-yl-ethyl)-acetamide FW 573.71

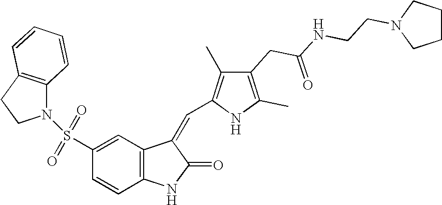

MS m/z 574 [M+1].

### Example 59

### 3-[1-{3,5-Dimethyl-4-[4-(4-methyl-piperazine-1-carbonyl)-phenyl]-1H-pyrrol-2-yl}-meth-(Z)-ylidene]-2-oxo-2,3-dihydro-1H-indole-5-sulfonic acid dimethylamide FW 547.68

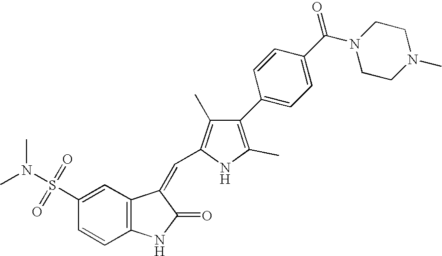

MS m/z 548 [M+1].

### Example 60

### 3-[1-{3,5-Dimethyl-4-[3-(morpholine-4-carbonyl)-phenyl]-1H-pyrrol-2-yl}-meth-(Z)-ylidene]-2-oxo-2,3-dihydro-1H-indole-5-sulfonic acid amide FW 506.58

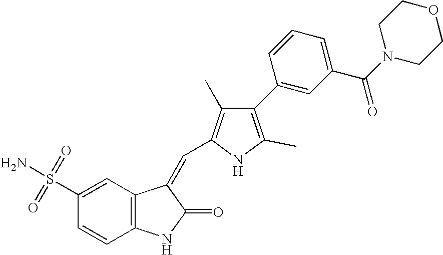

MS m/z 507 [M+1].

### Example 61

### 3-[1-[4-(4-Hydroxy-piperidin-1-ylmethyl)-3,5-dimethyl-1H-pyrrol-2-yl]-meth-(Z)-ylidene]-2-oxo-2,3-dihydro-1H-indole-5-sulfonic acid thiazol-2-ylamide FW 513.64

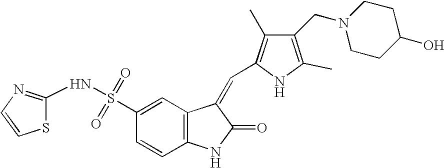

MS m/z 514 [M+1].

### Example 62

### 2-{5-[5-[(3-Chloro-phenyl)-methyl-sulfamoyl]-2-oxo-1,2-dihydro-indol-(3Z)-ylidenemethyl]-2,4-dimethyl-1H-pyrrol-3-yl}-N-(2-pyrrolidin-1-yl-ethyl)-acetamide FW 596.15

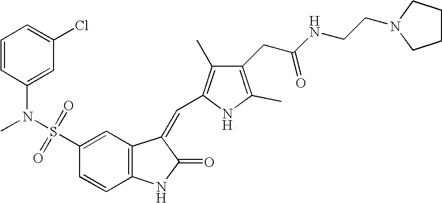

MS m/z 596 [M+1].

### Example 63

### N-(2-Diethylamino-ethyl)-2-{2,4-dimethyl-5-[2-oxo-5-sulfamoyl-1,2-dihydro-indol-(3Z)-ylidenemethyl]-1H-pyrrol-3-yl}-acetamide FW 473.60

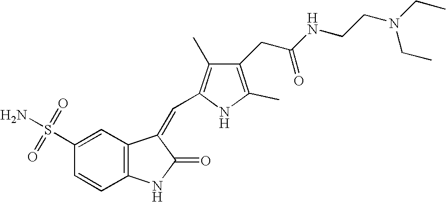

MS m/z 474 [M+1].

### Example 64

### N-(2-Diethylamino-ethyl)-2-{2,4-dimethyl-5-[5-methylsulfamoyl-2-oxo-1,2-dihydro-indol-(3Z)-ylidenemethyl]-1H-pyrrol-3-yl}-acetamide FW 487.63

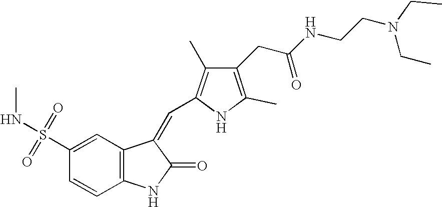

MS m/z 488 [M+1].

### Example 65

### 2-{2,4-Dimethyl-5-[2-oxo-5-sulfamoyl-1,2-dihydro-indol-(3Z)-ylidenemethyl]-1H-pyrrol-3-yl}-N-(2-morpholin-4-yl-ethyl)-acetamide FW 487.58

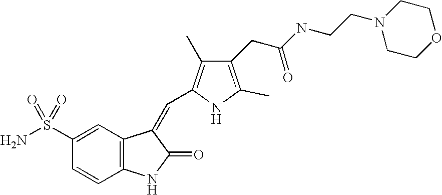

MS m/z 488 [M+1].

### Example 66

### 3-[1-{3,5-Dimethyl-4-[3-(morpholine-4-carbonyl)-phenyl]-1H-pyrrol-2-yl}-meth-(Z)-ylidene]-2-oxo-2,3-dihydro-1H-indole-5-sulfonic acid methylamide FW 520.61

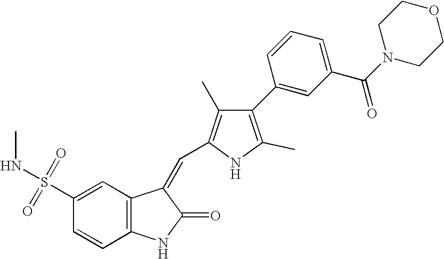

MS m/z 521 [M+1].

### Example 67

### 3-[1-{3,5-Dimethyl-4-[3-(morpholine-4-carbonyl)-phenyl]-1H-pyrrol-2-yl}-meth-(Z)-ylidene]-2-oxo-2,3-dihydro-1H-indole-5-sulfonic acid dimethylamide FW 534.64

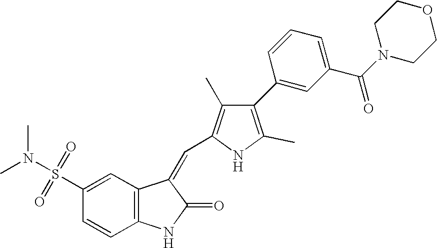

MS m/z 535 [M+1].

### Example 68

### 2-{5-[5-[(3-Chloro-phenyl)-methyl-sulfamoyl]-2-oxo-1,2-dihydro-indol-(3Z)-ylidenemethyl]-2,4-dimethyl-1H-pyrrol-3-yl}-N-(2-diethylamino-ethyl)-acetamide FW 598.17

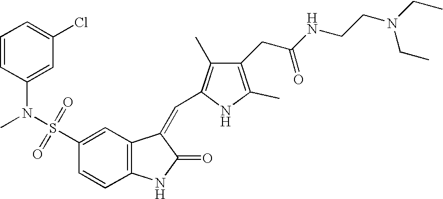

MS m/z 598 [M+1].

### Example 69

### 2-{2,4-Dimethyl-5-[5-methylsulfamoyl-2-oxo-1,2-dihydro-indol-(3Z)-ylidenemethyl]-1H-pyrrol-3-yl}-N-(2-morpholin-4-yl-ethyl)-acetamide FW 501.61

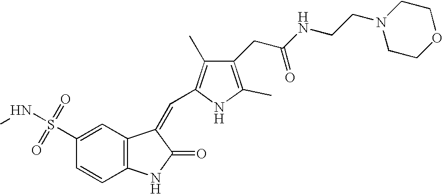

MS m/z 502 [M+1].

### Example 70

### 2-{5-[5-Dimethylsulfamoyl-2-oxo-1,2-dihydro-indol-(3Z)-ylidenemethyl]-2,4-dimethyl-1H-pyrrol-3-yl}-N-(2-morpholin-4-yl-ethyl)-acetamide FW 515.64

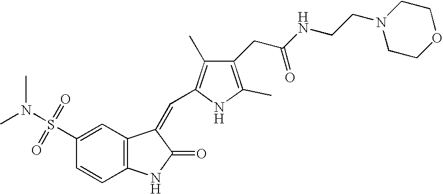

MS m/z 516 [M+1].

### Example 71

### 3-[1-(3,5-Diethyl-4-morpholin-4-ylmethyl-1H-pyrrol-2-yl)-meth-(Z)-ylidene]-2-oxo-2,3-dihydro-1H-indole-5-sulfonic acid amide FW 444.56

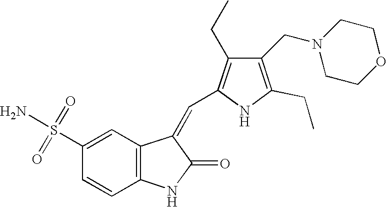

MS m/z 445 [M+1].

### Example 72

### 3-[1-{3,5-Dimethyl-4-[4-(morpholine-4-carbonyl)-phenyl]-1H-pyrrol-2-yl}-meth-(Z)-ylidene]-2-oxo-2,3-dihydro-1H-indole-5-sulfonic acid amide FW 506.58

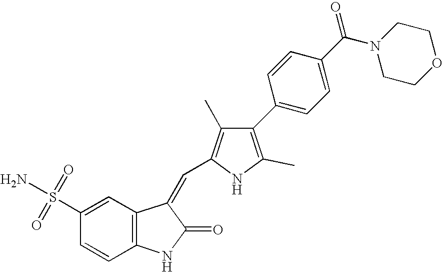

MS m/z 507 [M+1].

### Example 73

### 3-[1-{3,5-Dimethyl-4-[3-(4-methyl-piperazine-1-carbonyl)-phenyl]-1H-pyrrol-2-yl}-meth-(Z)-ylidene]-2-oxo-2,3-dihydro-1H-indole-5-sulfonic acid amide FW 519.63

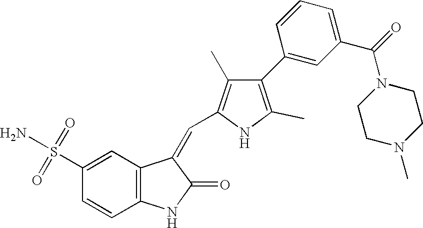

MS m/z 520 [M+1].

### Example 74

### 3-[1-{3,5-Dimethyl-4-[3-(4-methyl-piperazine-1-carbonyl)-phenyl]-1H-pyrrol-2-yl}-meth-(Z)-ylidene]-2-oxo-2,3-dihydro-1H-indole-5-sulfonic acid methylamide FW 533.65

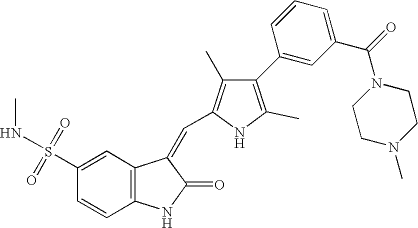

MS m/z 534 [M+1].

### Example 75

### N-(2-Diethylamino-ethyl)-2-{5-[5-dimethylsulfamoyl-2-oxo-1,2-dihydro-indol-(3Z)-ylidenemethyl]-2,4-dimethyl-1H-pyrrol-3-yl}-acetamide FW 501.65

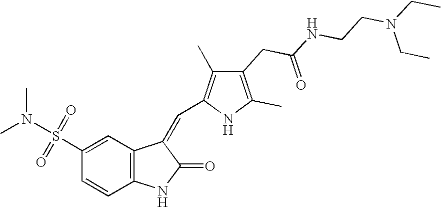

MS m/z 502 [M+1].

### Example 76

### 3-[1-{3,5-Dimethyl-4-[4-(morpholine-4-carbonyl)-phenyl]-1H-pyrrol-2-yl}-meth-(Z)-ylidene]-2-oxo-2,3-dihydro-1H-indole-5-sulfonic acid methylamide FW 520.61

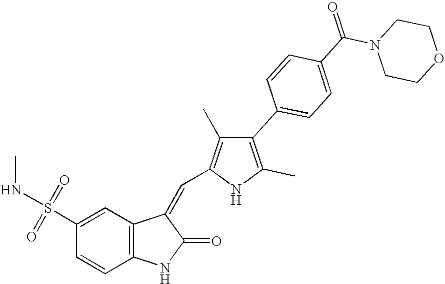

MS m/z 521 [M+1].

### Example 77

### 3-[1-{3,5-Dimethyl-4-[4-(morpholine-4-carbonyl)-phenyl]-1H-pyrrol-2-yl}-meth-(Z)-ylidene]-2-oxo-2,3-dihydro-1H-indole-5-sulfonic acid dimethylamide PHA710283 FW 534.64

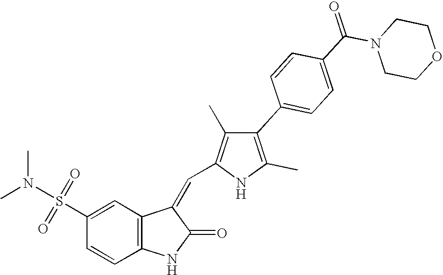

MS m/z 535 [M+1].

### Example 78

### 3-[1-{3,5-Dimethyl-4-[4-(4-methyl-piperazine-1-carbonyl)-phenyl]-1H-pyrrol-2-yl}-meth-(Z)-ylidene]-2-oxo-2,3-dihydro-1H-indole-5-sulfonic acid amide FW 519.63

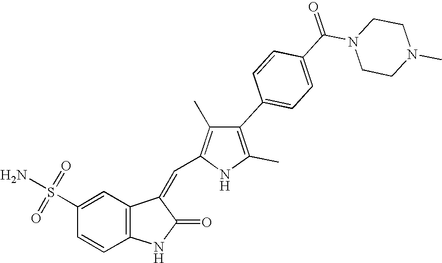

MS m/z 520 [M+1].

### Example 79

### 3-[1-{3,5-Dimethyl-4-[4-(4-methyl-piperazine-1-carbonyl)-phenyl]-1H-pyrrol-2-yl}-meth-(Z)-ylidene]-2-oxo-2,3-dihydro-1H-indole-5-sulfonic acid methylamide FW 533.65

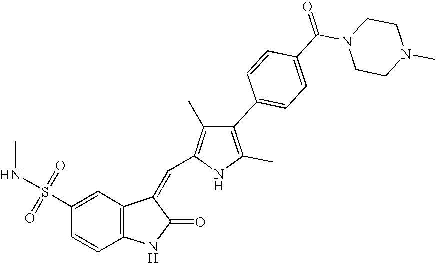

MS m/z 534 [M+1].

### Example 80

### 3-[1-{3,5-Dimethyl-4-[3-(4-methyl-piperazine-1-carbonyl)-phenyl]-1H-pyrrol-2-yl}-meth-(Z)-ylidene]-2-oxo-2,3-dihydro-1H-indole-5-sulfonic acid dimethylamide FW 547.68

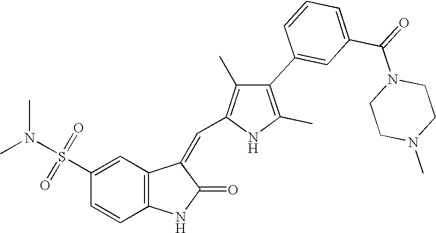

MS m/z 548 [M+1].

### Example 81

### 3-[1-(3-Nitro-1H-pyrrol-2-yl)-meth-(Z)-ylidene]-2-oxo-2,3-dihydro-1H-indole-5-sulfonic acid methylamide FW 348.34

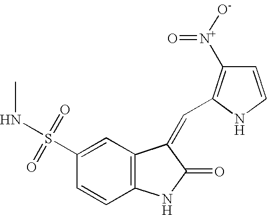

MS m/z 349 [M+1].

### Example 82

### 3-[1-(4-Nitro-1H-pyrrol-2-yl)-meth-(Z)-ylidene]-2-oxo-2,3-dihydro-1H-indole-5-sulfonic acid methylamide FW 348.34

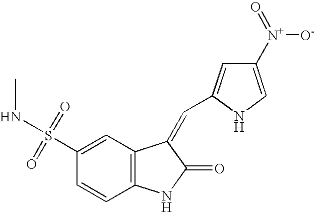

MS m/z 349 [M+1].

### Example 83

### 3-[2,4-Diethyl-5-(2-oxo-5-phenylsulfamoyl-1,2-dihydro-indol-3-ylidenemethyl)-1H-pyrrol-3-yl]-propionic acid

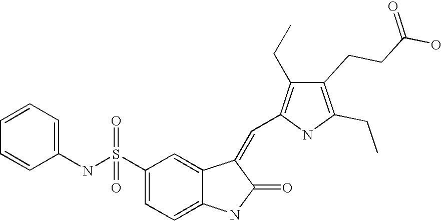

The above compound has been prepared according to commonly used method of organic synthesis known in the art, e.g., such as found in U.S. Pat. No. 6,395,734 and U.S. patent application Ser. No. 09/716,332.

### BIOLOGICAL EXAMPLES

### Example 1

### DNA-PK Assay Using 5-Sulfonamido Substituted Indolinones

The assay for DNA-PK was optimized to be performed in 96-well plates using a Scintillation Proximity Assay (SPA) format at room temperature.

The kinase reaction was performed in a total volume of 50 μl using 0.23 nM DNA-PKcs in a buffer containing 50 mM Tris, pH 7.4, 10 mM manganese chloride, 1 mM DTT, 3.6 nM activating DNA, 2.8 μM substrate peptide, 0.3 μM ATP, and 0.33 μCi γ³³P ATP. When inhibitors were included, they were diluted to 5× the desired concentration in 5% DMSO and diluted 1:5 in the reaction mixture bringing the final DMSO concentration to 1%. (The presence of 1% DMSO was shown to not inhibit the kinase reaction significantly.)

Activating DNA—DNA fragment consisting of 32 complementary base pairs with 5 non-complementary T's on both ends of the two strands Sequence—5′-TTTTT GGCCGCACGCGTCCACCATGGGGTACAACTACTTTTT-3′, and of the complementary strand—5′-TTTTTGTAGTTGTACCCCATGGTGGA CGCGTGCGGCCTTTTT-3′. Oligonucleotides were synthesized separately, annealed, and dialyzed against TE.

Peptide substrate—biotin-X-PESQEAFADLWPESQEAFADLWKKK. Dissolved in DMSO at a concentration of 5 mg/ml for the stock solution. Alternatively, fragments of the peptide substrate can be used, such as, without limitation, SQEAFADLW.

The kinase reaction was stopped by adding 200%1 of a solution containing 50 μM ATP, 5 mM EDTA, 0.1% Triton X-100, and 0.5 mg of streptavidin coated polyvinyltoluene SPA beads (Amersham). After a 10 min. incubation and a short spin (to pellet the beads), the plates were read on a Trilux plate reader.

The kinase reaction was found to be linear under these conditions for about 10 minutes.

The following compounds were found to inhibit DNA-PKcs in this assay:

| Compound | IC₅₀ (μM) |
|---|---|
| 3-{5-[(Z)-(5-iodo-2-oxo-1,2-dihydro-3H- | 1.5 |
| indol-3-ylidene)methyl]-4-methyl-1H- | |
| pyrrol-3-yl}propanoic acid | |
| 3-[2,4-Diethyl-5-(2-oxo-5- | 1.6 |
| phenylsulfamoyl-1,2-dihydro-indol-3- | |
| ylidenemethyl)-1H-pytrrol-3-yl]-propionic | |
| acid | |
| 2-oxo-3-[5-(2-pyrrolidin-1-yl-ethoxy-1H- | 1.7 |
| indol-2-ylmethylene]-2,3-dihydro-1H- | |
| indole-sulfonic acid amide | |
| 5-(5-bromo-2-oxo-1,2-dihydro-indol-3- | 34 |
| ylidenemethyl)-2,4-dimethyl-1H-pyrrole- | |
| 3-carboxylic acid-(2-diethylamino-ethyl)- | |
| amide | |
| 3-{3-(3-dimethylamino-propyl)-4,5,6,7- | 66 |
| tetrahydro-1H-indol-2-ylmethylene]-2-oxo- | |
| 2,3-dihydro-1H-indole-5-sulfonic acid | |
| methylamide | |

### Example 2

### Comparison of 3-[2,4-Diethyl-5-(2-oxo-5-phenylsulfamoyl-1,2-dihydro-indol-3-ylidenemethyl)-1H-pyrrol-3-yl]-propionic acid with Wortmannin in DNA-PK Assay

A. Materials

3-[2,4-Diethyl-5-(2-oxo-5-phenylsulfamoyl-1,2-dihydro-indol-3-ylidenemethyl)-1H-pyrrol-3-yl]-propionic acid was dissolved in DMSO at 20 mg/ml and stored at −70° C. Calicheamicin μl was a generous gift from George Ellestad (Wyeth-Ayers Research). The drug was dissolved at 2 mM in DMSO (Sigma) and stored at −70° C. Under these conditions, calicheamicin μl showed no loss of activity after 12 months. Wortmannin (Sigma) was dissolved at 1 mg/ml in DMSO and stored at −70° C. Recombinant p110 was purchased from Alexis and stored at −70° C. The glioblastoma cell line Mo59K and primary human fibroblasts was grown in Dulbecos MEM with glutamax (Invitrogen) supplemented with 10% Fetal calf serum, streptomycin and penicillin at 37° C. in a humidified cell incubator with 5% CO₂. FACS analysis of cell cycle distribution after propiumiodide staining was done using standard protocols. Monolayers were irradiated on ice using a Philips RT 100 X-ray machine. An acceleration voltage of 70 kV was used.

B. DNA-PK Assay

DNA-PKcs and Ku was purified from human placenta as previously described (13). DNA-PK kinase activity was measured by mixing 20-30 ng Ku, 3 ng DNA-PKcs with 0.5 mg/ml substrate peptide (EPPLSQEAFADLWKK) in a total volume of 10 μl DNA-PK kinase buffer (10 mM Tris-HC), pH 7.5, 0.1 mM EDTA, 50 mM NaCl, 20 mM MgCl2, 10 mM 2-mercaptoethanol, 62 μM ATP and 16 nM [³²P] ATP 5000 Ci/mmol (Amersham Pharmacia) and 20 ng of linear plasmid DNA (pBluescript KS+II linearized with Sma I). Inhibitors was diluted in PBS and added last. For data in FIG. 2A the DNA-PK cold ATP was omitted from the kinase buffer. The DNA-PK assay was performed with a fixed concentration of 3-[2,4-Diethyl-5-(2-oxo-5-phenylsulfamoyl-1,2-dihydro-indol-3-ylidenemethyl)-1H-pyrrol-3-yl]-propionic acid (0, 1, 2 and 4 μM) and various concentrations of cold ATP. The amount of [³²P] ATP was kept constant at 16 nM. All solutions and reaction, mixtures were kept on ice during preparation of the samples. The samples were incubated at 37° C. for 12 min and then transferred back to ice. Reactions were acidified by adding 10 μl of 50% acetic acid and then transferred to phosphocellulose filters (Whatman P81). Filters were washed in two changes of 250 ml of 15% acetic acid. Radioactivity bound to the filters was measured by scintillation counting. Samples with no added enzyme was used to determine the background that was subtracted from each value.

C. Measurement of DSB-Repair

The DSB analysis involves the following: the monolayer were rinsed with ice-cold PBS after exposure to drugs or X-rays or after one hour of repair at 37° C. at 5% CO₂ and the cells were scraped off the plastic plate. The suspension was mixed with agarose and transferred to a plug mold. The cells in the plug was lysed at 37° C. for 48 h in 1% Sodiumdodecylsulfate and 1 mg/ml proteinase K in 10 mM EDTA pH 8.2, and run in an agarose gel (0.7%) in 1×TAE at 18° C. for 17 hours, using constant field gel electrophoresis. The gel was stained with ethidium bromide and analyzed by a fluorescence scanner (Thyphoon, Amersham Pharmacia) and the fraction of DNA entering the gel (FAR) quantified using the imagequant software.

D. P110 Kinase Assay

Micelles containing phosphatidylinositol was prepared prior to each experiment by mixing 1 μg of phosphatidylinositol with 40 μg phosphatidylserine in 300 μl chloroform and the resulting solution dried under nitrogen gas. To the dried lipids was resuspended in 100 μl 25 mM Hepes pH 7.5, 1 mM EGTA by sonication. The kinase reaction was performed by mixing 2 μg of Recombinant p110 with 5 μl of micelles in 20 μl DNA-PK kinase buffer supplemented with the inhibitors of the invention and incubated at 37° C. for 30 minutes.

The reaction was stopped on ice and acidified by addition of 100 μl of 1 M HCl and Lipids was extracted with 300 μl methanol:chloroform (1:1). The radioactivity in the methanol:chloroform phase was measured by liquid scintillation. Samples with no added p110 was used to determine background that typically was 100-fold below maximal activity. Relative kinase activity was calculated on background subtracted values.

E. H2AX Phosphorylation

Confluent monolayers of HeLa cells in 1.5 cm plates were preincubated or not preincubated with the inhibitors indicated in FIG. 5 for 3 h at 37° C. before addition of calicheamicin μl (10 nM) and further incubated at 37° C. for 45 minutes to allow induction of DSB. After washing with cold PBS cells were scraped off the plates, and lysed in 500 μl cell lysis buffer (10 mM EDTA, 20 mM Tris, 1% Nonidet P-40, PH 8, 10 mM NaF, 1 mM Na VO₄, 1 mM Na Mo₄, Protease inhibitors and 200 μl of (100 μM) Okadaic acid) and nuclear pellet collected by centrifugation. Histones were acid extracted in 3 volumes of extraction buffer (0.5 M HCL+10% glycerol+0.1M mercaptethanol), precipitated with trichloro acetic acid (25%) and separated on 15% SDS-PAGE and transferred to PDF-membrane. The membrane was blocked with blotto (5% non-fat milk, 10 mM tris-HCl pH7.5 140 mM NaCl, 0.05 NP-40) and incubated with an antibody specific for the phosphorylated form of H2AX (Upstate Bioechnology) diluted 1:2000 in blotto. After incubation with secondary antibody the membrane was developed with ECL (Pierce) and analyzed on a video based system (Chemidoc, Biorad).

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains. All patents and publications are herein incorporated by reference to the same extent as if each individual publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group. For example, if X is described as selected from the group consisting of bromine, chlorine, and iodine, claims for X being bromine and claims for X being bromine and chlorine are fully described.

Other embodiments are within the following claims.

1. A method of inhibiting DNA-PK comprising administering to a patient in need of such inhibition, an effective amount of a 5-sulfonamido-substituted indolinone.

2. The method of claim 1, wherein the patient is administered a compound of Formula (I): 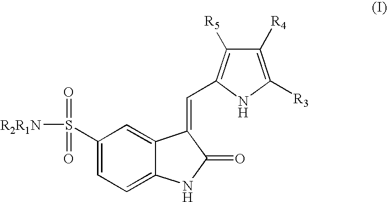
wherein:
R₁ and R₂ are independently selected from the group consisting of H, optionally substituted phenyl, thiazolyl and lower alkyl,
or R₁ and R₂ combine to form an optionally fused heterocyclic ring, which is optionally substituted by —O-alkyl, Br, Cl or F, provided that only one of R₁ and R₂ is alkyl or hydrogen at the same time and further provided that R₁ is not alkyl when R₂ is hydrogen and that R₁ is not hydrogen when R₂ is alkyl;
R₃, R₄ and R₅ are independently selected from the group consisting of H, lower alkyl optionally substituted with hydroxy and —(Y)₀₋₁—Y₁,
or R₃ and R₄ may combine to form a cyclic 6-membered alicyclic ring which may be substituted with one or more lower alkyl, provided that no more than two of R₃, R₄ or R₅ are H at the same time and further provided that at least one of R₃, R₄ or R₅ is —(Y)₀₋₁—Y₁;
Y is —CH₂—, —CH₂—CH₂—, —CH₂—CH₂—CH₂— or —C(O)NHR₆—;
Y₁ is —C(O)OR′, —C(O)NR₆R₇ or —NR₆R₇, where R′ is H or lower alkyl;
R₆ and R₇ are independently selected from the group consisting of H and lower alkyl optionally substituted by —NR₈R₉;
or R₆ and R₇ may combine to form a heterocyclic ring which may include an additional heteroatom selected from the group consisting of N, O and S and which may be further substituted by lower alkyl or hydroxy;
R₈ and R₉ are independently H and lower alkyl;
or R₈ and R₉ may combine to form a heterocyclic ring which may include an additional heteroatom selected from the group consisting of N, O and S and pharmaceutically acceptable salts thereof.

3. The method of claim 1, wherein the patient is administered a compound of Formula (II): 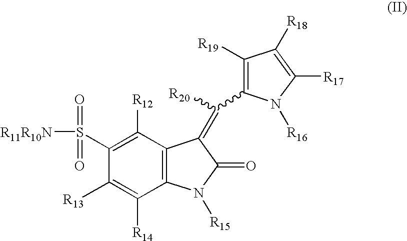
wherein:
R₁₀ and R₁₁ are independently selected from the group consisting of hydrogen, alkyl optionally substituted with amino, hydroxy, a 5-membered to 6-membered heteroalicyclic ring or halo, aryl, heteroaryl, cycloalkyl, alkenyl, alkynyl, heteroalicyclic, or R₁₀ and R₁₁ may combine to form a 5-membered or 6-membered heterocyclic ring which may be optionally fused;
R₁₂, R₁₃ and R₁₄ are independently selected from the group consisting of hydrogen, alkyl, trihaloalkyl, cycloalkyl, alkenyl, alkynyl, aryl, heteroaryl, heteroalicyclic, hydroxy, alkoxy, aryloxy, mercapto, alkylthio, arylthio, sulfinyl, sulfonyl, S-sulfonamido, N-sulfonamido, trihalomethane-sulfonamido, carbonyl, C-carboxy, O-carboxy, C-amido, N-amido, cyano, nitro, halo, O-carbamyl, N-carbamyl, O-thiocarbamyl, N-thiocarbamyl, amino and —NR₂₁R₂₂;
R₁₅ is selected from the group consisting of hydrogen, alkyl, alkenyl, alkynyl, cycloalkyl, aryl, hydroxy, alkoxy, C-carboxy, O-carboxy, acetyl, C-amido, C-thioamido, sulfonyl and trihalomethanesulfonyl;
R₂₀ is selected from the group consisting of hydrogen, halo, alkyl, cycloalkyl, aryl, heteroaryl and heteroalicyclic;
R₂₁ and R₂₂ are independently selected from the group consisting of hydrogen, alkyl, cycloalkyl, aryl, carbonyl, acetyl, sulfonyl, trifluoromethanesulfonyl and, combined, a five- or six-member heteroalicyclic ring;
R₁₃ and R₁₄ may combine to form a six-member aryl ring, a methylenedioxy group or an ethylenedioxy group;
R₁₆ is selected from the group consisting of hydrogen, alkyl, cycloalkyl, alkenyl, alkynyl, aryl, heteroaryl, heteroalicyclic, hydroxy, alkoxy, aryloxy, carbonyl, acetyl, C-amido, C-thioamido, amidino, C-carboxy, O-carboxy, sulfonyl and trihalomethane-sulfonyl;
R₁₇, R₁₈ and R₁₉ are independently selected from the group consisting of hydrogen, alkyl, trihaloalkyl, cycloalkyl, alkenyl, alkynyl, aryl, heteroaryl, heteroalicyclic, hydroxy, alkoxy, aryloxy, mercapto, alkylthio, arylthio, sulfinyl, sulfonyl, S-sulfonamido, N-sulfonamido, carbonyl, C-carboxy, O-carboxy, cyano, nitro, halo, O-carbamyl, N-carbamyl, O-thiocarbamyl, N-thiocarbamyl, C-amido, N-amido, amino, —NR₂₁R₂₂ and -(alk₁)Z wherein Alk₁ is selected from the group consisting of alkyl, alkenyl or alkynyl; and, Z is a polar group;
or R₁₇ and R₁₈ or R₁₈ and R₁₉ may combine to form an alicyclic ring;
or a pharmaceutically acceptable salt thereof.

4. The method of claim 1, wherein the patient is administered a compound of Formula (III): 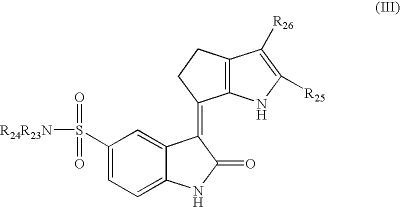
wherein:
R₂₃ and R₂₄ are independently selected from the group consisting of H, optionally substituted phenyl, lower alkyl and cycloalkyl,
or R₂₃ and R₂₄ combine to form an optionally fused heterocyclic ring, which is optionally substituted by —O-alkyl, Br, Cl or F;
R₂₅ and R₂₆ are independently selected from the group consisting of hydrogen, lower alkyl, sulfonyl, —C(O)R₂₇, —C(O)OR₂₇, —C(O)NR₂₇R₂₈, halo, trihaloalkyl, aryl, heteroaryl, wherein R₂₇ and R₂₈ are independently selected from the group consisting of hydrogen, lower alkyl, lower alkyl substituted with one or more of amino, hydroxy, 5-membered to 6-membered heterocyclic ring or R₂₇ and R₂₈ may combine to form a 5-membered to 6-membered heterocyclic ring which may be optionally substituted with alkyl;
or a pharmaceutically acceptable salt thereof.

5. The method of claim 1, wherein the patient is administered a compound selected from the group consisting of Formula (IV), (V) and (VI): 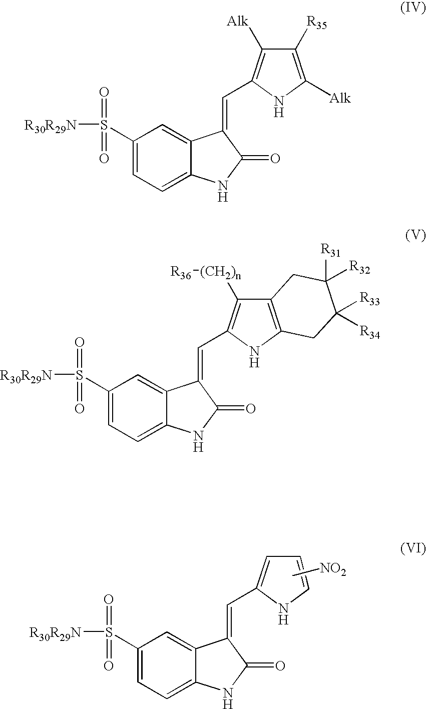
wherein:
R₂₉-R₃₄ are independently selected from the group consisting of H, lower alkyl and cycloalkyl;
R₃₅ is selected from the group consisting of —CH₂—C(O)—X′—(CH₂)ₙ—R₃₆, —CH₂—R₃₆ 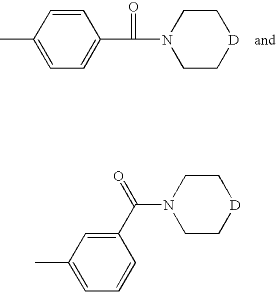
where D is O or N—CH₃;
X′ is NH, S, O or a bond;
R₃₆ is a polar group selected from the group consisting of —C(O)OR′, —C(O)NR₃₇R₃₈, piperazinyl and morpholinyl, Z may be further substituted by —(CH₂)₀₋₁-Z₁, where Z₁ is a polar group selected from the group consisting of —C(O)OR₃₇, —C(O)NR₃₇R₃₈, amino, dialkylamino, hydroxy, piperazinyl, pyrrolidinyl and morpholinyl; when R₃₆ is further substituted, R₃₇ is not present;
R₃₇ and R₃₈ are independently H and lower alkyl;
n is 0-2; and
Alk is lower alkyl of 1-4 carbons;
and pharmaceutically acceptable salts thereof.

6. The method of claim 3, wherein R¹⁰ is phenyl, R₁₂, R₁₃, R₁₄, R₁₅, R₁₆ and R₂₀ are hydrogen, R₁₇ and R₁₉ are ethyl and R₁₈ is a propionic acid moiety.

7. The method of claim 1, wherein the compound administered is 3-[2,4-Diethyl-5-(2-oxo-5-phenylsulfamoyl-1,2-dihydro-indol-3-ylidenemethyl)-1H-pyrrol-3-yl]-propionic acid.

8. The method of claim 1, wherein the patient undergoing such treatment has cancer.

9. The method of claim 8, wherein the cancer is selected from the group consisting of squamous cell carcinoma, astrocytoma, Kaposi's sarcoma, glioblastoma, lung cancer, bladder cancer, head and neck cancer, melanoma, ovarian cancer, prostate cancer, breast cancer, small-cell lung cancer, glioma, colorectal cancer, genitourinary cancer and gastrointestinal cancer.

10. An assay for determining the inhibition of DNA-PK kinase comprising the steps of:
(a) contacting purified DNA-PK with a peptide substrate inhibitor, double stranded activating DNA and appropriate buffer for a sufficient period of time to allow phosphorylation of the substrate;
(b) measuring substrate phosphorylation

11. The method of claim 10, wherein the double stranded activating DNA consists of an oligonucleotide annealed from oligonucleotides with the sequence of:
(SEQ ID NO:1)
5′-TTTTTGGCCGCACGCGTCCACCATGGGGTACAACTACTTTTT-3′
and
(SEQ ID NO:2)
5′-TTTTTGTAGTTGTACCCCATGGTGGACGCGTGCGGCCTTTTT-3′.

12. The method of claim 10, wherein the sequence of the peptide substrate is EPPLSQEAFADLWKK (SEQ ID NO: 3).

13. The method of claim 10, wherein the sequence of the peptide substrate is biotin-X-PESQEAFADLWKK (SEQ ID NO: 4).

14. The method of claim 10, wherein the sequence of the peptide substrate is SQEAFADLWKK (SEQ ID NO: 5).

15. The method of claim 10, wherein substrate phosphorylation is measured by a scintillation proximity assay.

16. The method of claim 10, wherein substrate phosphorylation is measured by capture on a filter and scintillation counting.

17. A compound according to the following formula: [chemical structure]
